(19) United States
(12) Patent Application Publication
(10) Pub. No.: US 2023/0241075 A1
(43) Pub. Date: Aug. 3, 2023

(54) IRAK DEGRADERS AND USES THEREOF

(71) Applicant: Kymera Therapeutics, Inc., Watertown, MA (US)

(72) Inventors: Veronica CAMPBELL, Watertown, MA (US); Alice MCDONALD, Watertown, MA (US); Jared GOLLOB, Watertown, MA (US); Anthony SLAVIN, Watertown, MA (US)

(21) Appl. No.: 18/002,116

(22) PCT Filed: Jun. 17, 2021
(86) PCT No.: PCT/US2021/037952

§ 371 Date: Dec. 16, 2022

Related U.S. Application Data
(60) Provisional application No. 63/089,398, filed on Oct. 8, 2020.
(60) Provisional application No. 63/070,022, filed on Aug. 25, 2020.
(60) Provisional application No. 63/040,407, filed on Jun. 17, 2020.

(51) Int. Cl. A61K 31/5386; A61K 31/4545; A61P 17/10

(52) U.S. Cl. CPC A61K 31/5386; A61K 31/4545; A61P 17/10

(57) ABSTRACT

The present invention provides methods for identifying or selecting a patient having an elevated level of an inflammatory biomarker, and methods for treating a disease or disorder in a patient comprising selecting a patient having an elevated level of an inflammatory biomarker, and administering to the patient an IRAK degrader.

### TECHNICAL FIELD OF THE INVENTION

The present invention relates to compounds for modulation of one or more interleukin-1 receptor-associated kinases (IRAK) via ubiquitination and/or degradation, and uses thereof for treating a disease or disorder.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(0:40-46).

An ongoing need exists in the art for effective treatments for disease, especially hyperplasias and cancers, such as multiple myeloma. However, non-specific effects, and the inability to target and modulate certain classes of proteins altogether, such as transcription factors, remain as obstacles to the development of effective anti-cancer agents. As such, small molecule therapeutic agents that leverage E3 ligase mediated protein degradation to target cancer-associated proteins such as interleukin-1 receptor-associated kinases (“IRAK”) hold promise as therapeutic agents. Accordingly, there remains a need to find compounds that are IRAK degraders useful as therapeutic agents.

### SUMMARY OF THE INVENTION

As described herein, the inventors have discovered that levels of certain inflammatory biomarkers in hidradenitis suppurativa (HS) and atopic dermatitis (AD) patients are indicative of patient responsiveness to treatment with IRAK degraders, including, for example, those as described herein. The inflammatory biomarkers can be cutaneous and circulating inflammatory biomarkers. As shown herein, certain inflammatory biomarkers levels can be used, for example, for selecting patients for a treatment using IRAK degraders.

Accordingly, in one aspect, the present invention provides a method of identifying or selecting a patient having an elevated level of an inflammatory biomarker, comprising measuring a level of an inflammatory biomarker in a sample of a patient, and selecting a patient having an elevated level of an inflammatory biomarker in the sample. In some embodiments, a patient is a hidradenitis suppurativa patient. In some embodiments, a patient is an atopic dermatitis patient.

In another aspect, the present invention provides a method of identifying or selecting a patient having a reduced level of an inflammatory biomarker after a treatment with an IRAK degrader, comprising measuring a level of an inflammatory biomarker in a sample of a patient after a treatment with an IRAK degrader, and selecting a patient having a reduced level of an inflammatory biomarker in a sample. In some embodiments, a patient is a hidradenitis suppurativa patient. In some embodiments, a patient is an atopic dermatitis patient.

In another aspect, the present invention provides a method of treating a disease or disorder in a patient having an elevated level of an inflammatory biomarker, comprising administering to the patient a therapeutically effective amount of an IRAK degrader. In some embodiments, a disease or disorder is hidradenitis suppurativa. In some embodiments, a disease or disorder is an atopic dermatitis.

In another aspect, the present invention provides a method of treating a disease or disorder in a patient having a reduced level of an inflammatory biomarker after a treatment with an IRAK degrader, comprising administering to the patient a therapeutically effective amount of an IRAK degrader. In some embodiments, a disease or disorder is hidradenitis suppurativa. In some embodiments, a disease or disorder is an atopic dermatitis.

In another aspect, the present invention provides a method of treating a disease or disorder in a patient, comprising selecting a patient having an elevated level of an inflammatory biomarker, and administering to the patient a therapeutically effective amount of an IRAK degrader. In some embodiments, a disease or disorder is hidradenitis suppurativa. In some embodiments, a disease or disorder is an atopic dermatitis.

In another aspect, the present invention provides a method of treating a disease or disorder in a patient, comprising selecting a patient having a reduced level of an inflammatory biomarker after a treatment with an IRAK degrader, and administering to the patient a therapeutically effective amount of an IRAK degrader. In some embodiments, a disease or disorder is hidradenitis suppurativa. In some embodiments, a disease or disorder is an atopic dermatitis.

In some embodiments, an inflammatory biomarker is selected from those as described herein. In some embodiments, an IRAK degrader is selected from those as described herein.

### BRIEF DESCRIPTION OF FIGURES

FIG. 1 depicts potent and selective degraders of IRAK4.

FIG. 2 depicts orally active IRAK4 degrader blocks IL-1 driven neutrophilic infiltration in MSU air pouch model.

FIG. 3 depicts full degradation of IRAK4 in skin and lymphoid tissues in higher species.

FIG. 4 depicts that IRAK4 degradation has broader and more potent effect on TIR activation compared to kinase inhibition.

FIG. 5 depicts that IRAK4 degradation reduces skin thickening and inhibits cytokine signaling in imiquimod induced psoriasis mouse model.

FIG. 6 depicts HS patient responses to Degrader 2 ex vivo in PBMC subsets.

FIG. 7 depicts IRAK signal at baseline and following ex vivo treatment with Degrader 2 in HS patients.

FIG. 8 depicts IRAK4 immunofluorescence (IF) of patient biopsies (A) and cell count by intensity per biopsy location (B).

FIG. 9 depicts absolute quantification of IRAK4 by mass spectrometry (MS) in patient biopsies normalized to PARK7.

FIG. 10 shows IRAK4 expression in peripheral blood mononuclear cells is highest in monocytes.

FIG. 11 shows that IRAK4 degrader downregulates IRAK4 expression across all PBMC subsets with comparison to IRAK4 inhibitor.

FIG. 12 shows methods for measuring IRAK4 protein and pro-inflammatory gene transcripts in HS skin biopsies (A) and healthy subject skin/monocytes (B).

FIG. 13 shows IRAK4 protein expression is elevated in HS skin compared to skin from healthy subjects.

FIG. 14 shows IRAK4 is upregulated in dermis and epidermis of HS patients relative to skin of healthy subjects.

FIG. 15 depicts transcriptional profiling which shows clear differences between HS skin biopsy sites, but not across disease severity.

FIG. 16 shows transcripts for multiple mediators of inflammation are upregulated in HS skin lesions.

FIG. 17 shows multiple proinflammatory transcripts correlate with IRAK4 protein levels in HS skin lesions.

FIG. 18 shows IRAK4 Degrader 2 inhibits TLR-mediated induction of HS-overexpressed proinflammatory transcripts in healthy monocytes.

### DETAILED DESCRIPTION OF THE INVENTION

### 1. General Description of Certain Embodiments of the Invention

As shown herein, it has been found that there is a correlation between levels of certain inflammatory biomarkers in hidradenitis suppurativa and atopic dermatitis patients and the likelihood of responsiveness to treatment with an IRAK degrader. Without wishing to be bound by any particular theory, the inventors have discovered that hidradenitis suppurativa and atopic dermatitis patients having an elevated level of certain inflammatory biomarkers, for example, as those described herein, are more likely to benefit from an IRAK degrader treatment.

In one aspect, the present invention provides a method of identifying or selecting a patient having an elevated level of an inflammatory biomarker, comprising measuring a level of an inflammatory biomarker in a sample of a patient, and selecting a patient having an elevated level of an inflammatory biomarker in the sample. In some embodiments, a patient is a hidradenitis suppurativa patient. In some embodiments, a patient is an atopic dermatitis patient.

In another aspect, the present invention provides a method of identifying or selecting a patient having a reduced level of an inflammatory biomarker after a treatment with an IRAK degrader, comprising measuring a level of an inflammatory biomarker in a sample of a patient after a treatment with an IRAK degrader, and selecting a patient having a reduced level of an inflammatory biomarker in a sample. In some embodiments, a patient is a hidradenitis suppurativa patient. In some embodiments, a patient is an atopic dermatitis patient.

In another aspect, the present invention provides a method of treating a disease or disorder in a patient having an elevated level of an inflammatory biomarker, comprising administering to the patient a therapeutically effective amount of an IRAK degrader. In some embodiments, a disease or disorder is hidradenitis suppurativa. In some embodiments, a disease or disorder is an atopic dermatitis.

In another aspect, the present invention provides a method of treating a disease or disorder in a patient having a reduced level of an inflammatory biomarker after a treatment with an IRAK degrader, comprising administering to the patient a therapeutically effective amount of an IRAK degrader. In some embodiments, a disease or disorder is hidradenitis suppurativa. In some embodiments, a disease or disorder is an atopic dermatitis.

In another aspect, the present invention provides a method of treating a disease or disorder in a patient, comprising selecting a patient having an elevated level of an inflammatory biomarker, and administering to the patient a therapeutically effective amount of an IRAK degrader. In some embodiments, a disease or disorder is hidradenitis suppurativa. In some embodiments, a disease or disorder is an atopic dermatitis.

In another aspect, the present invention provides a method of treating a disease or disorder in a patient, comprising selecting a patient having a reduced level of an inflammatory biomarker after a treatment with an IRAK degrader, and administering to the patient a therapeutically effective amount of an IRAK degrader. In some embodiments, a disease or disorder is hidradenitis suppurativa. In some embodiments, a disease or disorder is an atopic dermatitis.

In some embodiments, an inflammatory biomarker level in a sample is measured using a method as described herein. In some embodiments, an inflammatory biomarker is selected from those as described herein. In some embodiments, an IRAK degrader is selected from those as described herein.

### 2. Definitions

As used herein, the term “an IRAK degrader” refers to an agent that degrades IRAK, including IRAK1, IRAK2, IRAK3, and/or IRAK4. Various IRAK degraders have been described previously, for example, in WO 2019/133531 and WO 2020/010227, the contents of each of which are incorporated herein by reference in their entireties. In some embodiments, an IRAK degrader is a heterobifunctional compound that binds to and/or inhibits both an IRAK kinase and an E3 ligase with measurable affinity resulting in the ubiquitination and subsequent degradation of IRAK. In certain embodiments, an IRAK has an DC50 of less than about 50 μM, less than about 1 μM, less than about 500 nM, less than about 100 nM, less than about 10 nM, or less than about 1 nM.

As used herein, the term “an inflammatory biomarker” refers characteristic biological properties or molecules that can be detected and measured in parts of the body, including, for example, the blood or a tissue. An inflammatory biomarker can indicate either normal or diseased processes in the body. An inflammatory biomarker can be a specific cell, molecule, gene, gene product, enzyme, or hormone.

As used herein, Degrader 1 is an IRAK4 degrader of structure

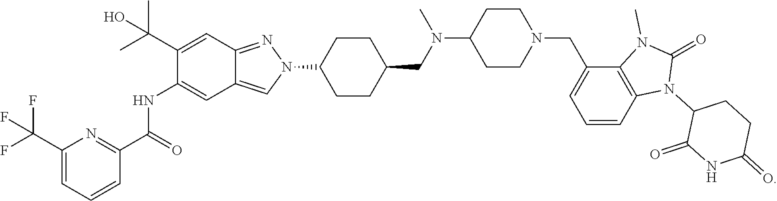

Degrader 2 is an IRAK4 degrader of structure

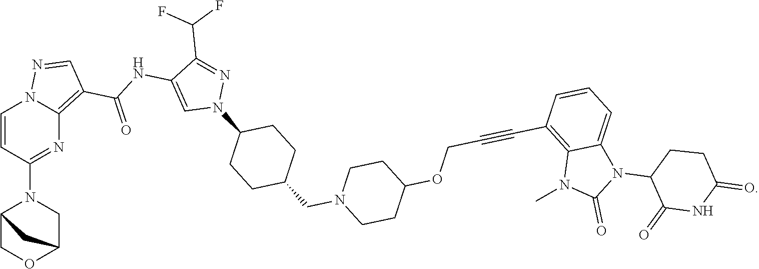 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

As used herein, “ELISA” or “enzyme-linked immunosorbent assay” is an immunoassay known in the art for detecting the presence of analytes in liquid samples. There are many types of ELISA methods, including, but not limited to, direct ELISAs, indirect ELISAs, sandwich ELISAs, competitive ELISAs, multiplex ELISAs, ELISPOT technologies, and other similar techniques known in the art. Principles of these immunoassay methods are known in the art, for example John R. Crowther, The ELISA Guidebook, 1st ed., Humana Press 2000, ISBN 0896037282, the contents of which are incorporated herein by reference in their entirety. Typically, ELISAs are performed with antibodies, but they can be performed with any capture agents that bind specifically to one or more inflammatory biomarkers, that can then be detected.

As used herein, the terms “inhibits,” “decreases,” “lowers,” or “reduces” are used interchangeably and encompass any measurable decrease in biological function and/or activity and/or concentration. For example, in some embodiments, an IRAK degrader described herein inhibits or reduces IRAK function and/or activity in a given system or assay or subject by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or 100%, relative to a control or baseline amount of that function and/or activity.

As used herein, the term “elevated level” of a substance (e.g., an inflammatory biomarker) in a sample refers to an increase in the amount or concentration of the substance of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to the amount or concentration of the substance in a control sample or control samples. A subject can also be determined to have an “elevated level” of a substance if the amount or concentration of the substance is increased by one standard deviation, two standard deviations, three standard deviations, four standard deviations, five standard deviations, or more relative to the mean (average) or median amount or concentration of the substance in a control group of samples or a baseline group of samples or a retrospective analysis of patient samples.

As used herein, the term “reduced level” or “lowered level” of a substance (e.g., an inflammatory biomarker) in a sample refers to a decrease in the amount or concentration of the substance of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or 100% relative to the amount or concentration of the substance in a control sample or control samples. A subject can also be determined to have an “reduced level” or “lowered level” of a substance if the amount or concentration of the substance is decreased by about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or more, relative to the amount or concentration of the substance in a control sample or control samples. A subject can also be determined to have an “reduced level” or “lowered level” of a substance if the amount or concentration of the substance is decreased by one standard deviation, two standard deviations, three standard deviations, four standard deviations, five standard deviations, or more relative to the mean (average) or median amount or concentration of the substance in a control group of samples or a baseline group of samples or a retrospective analysis of patient samples.

As used herein, the terms “control sample” or “control samples” refer to a sample of an individual or samples of a group of individuals, respectively, who are not suffering from the disease or disorder (e.g., hidradenitis suppurativa and/or atopic dermatitis), or an internal control, as determined by techniques known in the art. In some embodiments, a control or baseline level is previously determined, or measured prior to the measurement in the sample, or obtained from a database of such control samples. In some embodiments, a control sample and a subject sample are not tested simultaneously. In some embodiments, a control sample refers to an untreated sample (or treated with a negative control, such as a solvent) of an individual, who suffers from the disease or disorder (e.g., hidradenitis suppurativa and/or atopic dermatitis).

As used herein, the terms “treatment,” “treat,” and “treating” refer to preventing, reversing, alleviating, reducing the severity of, delaying the onset of, or inhibiting the progress of a disease or disorder, or one or more symptoms thereof, as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example to prevent or delay their recurrence.

As used herein, the term “patient” refer to an animal, preferably a mammal, and most preferably a human.

As used herein, the term “a therapeutically effective amount of” refers to the amount of an IRAK degrader, which measurably reduces the amount of IRAK. As used herein, the term “measurably reduce” refers to a measurable change in the amount or concentration of IRAK, between a sample comprising an IRAK degrader described herein, or a salt or a composition thereof, and an equivalent sample in the absence of said IRAK degrader, or a salt or composition thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. 100541 Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

### 3. Description of Exemplary Methods and Uses

In one aspect, the present invention provides a method of measuring an inflammatory biomarker level in a patient, comprising measuring an inflammatory biomarker level in a sample of the patient.

In one aspect, the present invention provides a method of identifying or selecting a patient having an elevated level of an inflammatory biomarker, comprising measuring a level of an inflammatory biomarker in a sample of a patient, and selecting a patient having an elevated level of an inflammatory biomarker in a sample.

In one aspect, the present invention provides a method of identifying or selecting a patient having a reduced level of an inflammatory biomarker after a treatment with an IRAK degrader, comprising measuring a level of an inflammatory biomarker in a sample of a patient after a treatment with an IRAK degrader, and selecting a patient having a reduced level of an inflammatory biomarker in a sample. In some embodiments, the present invention provides a method of identifying or selecting a patient having a reduced level of an inflammatory biomarker after a treatment with an IRAK degrader, comprising administering to a patient a therapeutically effective amount of an IRAK degrader, measuring a level of an inflammatory biomarker in a sample of a patient after a treatment with an IRAK degrader, and selecting a patient having a reduced level of an inflammatory biomarker in a sample after a treatment with an IRAK degrader.

In some embodiments, a patient is a hidradenitis suppurativa patient. In some embodiments, a hidradenitis suppurativa patient has active mild, moderate, or severe hidradenitis suppurativa. In some embodiments, active mild, moderate, or severe hidradenitis suppurativa is determined by an HS-PGA assessment. In some embodiments, a patient is an atopic dermatitis patient. In some embodiments, an atopic dermatitis patient has active moderate or severe atopic dermatitis. In some embodiments, active moderate or severe atopic dermatitis is determined by a PGA assessment.

In some embodiments, a patient is not, or has not been, on a biologic or other immunosuppressive treatment for HS or AD. In some embodiments, a patient is not, or has not been, on a biologic treatment for HS or AD within 3 months or 5 half-lives, whichever is longer. In some embodiments, a patient is not, or has not been, on a non-biologic immunosuppressive treatment (eg. Cyclosporin) within 4 weeks.

In some embodiments, a sample of a patient is a blood sample. In some embodiments, a sample of a patient is a skin sample. In some embodiments, a sample of a patient is a serum sample. In some embodiments, a sample of a patient is a plasm sample. In some embodiments, a sample of a patient is a peripheral blood sample. In some embodiments, a skin sample is a lesional skin sample. In some embodiments, a skin sample is a peri-lesional skin sample. In some embodiments, a skin sample is a non-lesional sample.

The level of an inflammatory biomarker can be measured by a variety of methods, such as those described herein. In some embodiments, measuring a level of an inflammatory biomarker in a sample comprises using an ELISA method. In some embodiments, measuring a level of an inflammatory biomarker in a sample comprises using a flow cytometry-based method. In some embodiments, measuring a level of an inflammatory biomarker in a sample comprises using a Western blots method. In some embodiments, measuring a level of an inflammatory biomarker in a sample comprises using an immunoprecipitation method. In some embodiments, measuring a level of an inflammatory biomarker in a sample comprises using a dot blotting method. In some embodiments, measuring a level of an inflammatory biomarker in a sample comprises using an immunohistochemistry method. In some embodiments, measuring a level of an inflammatory biomarker in a sample comprises using an immunofluorescence method. In some embodiments, measuring a level of an inflammatory biomarker in a sample comprises using a radioimmunoassay method. In some embodiments, measuring a level of an inflammatory biomarker in a sample comprises using a method selected from those described in the Examples.

In some embodiments, an inflammatory biomarker is a cytokine. In some embodiments, an inflammatory biomarker is a cytokine produced by macrophage. In some embodiments, an inflammatory biomarker is a cytokine produced by T lymphocytes (T-cells). In some embodiments, a cytokine is selected from IL1b, IL-6, IL-8, IL-12p70, IL22, IL-23, TNFa, IL-18, IL-17A, IL-17F, IL-19, INFy, IL-27, IL36a, IL-36b, IL-36y, M-CSF, GM-CSF, IL-10, sTNFRI, G-CSF, CXCL1, CCL3, IL-4, IL-5, IL-13, TSLP, IL-33, IL-25, IL-31, and IL-9. In some embodiments, a cytokine is a pro-inflammatory (inflammation-promoting) cytokine, including, for example, IL-1α, IL-1β, IL-2, IL-6, Il-8, IL-12, TNF-α, an dIFN-γ. In some embodiments, a cytokine is an anti-inflammatory (inflammation-suppressive) cytokine, including, for example, IL-4, IL-5, IL-10, TGF-β. In some embodiments, a cytokine is IL-5. In some embodiments, a cytokine is IL-7. In some embodiments, measuring a cytokine level in a sample of a patient comprises using a cultured peripheral blood mononuclear cell (PBMC) assay. In some embodiments, measuring a cytokine level in a sample of a patient comprises using an ELISA method. In some embodiments, measuring a cytokine level in a sample of a patient comprises using a multiplex bead assay.

In some embodiments, an inflammatory biomarker is an immune-related effector. In some embodiments, an immune-related effector is a leukocyte. In some embodiments, a leukocyte is selected from granulocytes (neutrophils, basophils, eosinophils) monocytes, macrophages, dendritic cells, and lymphocytes (B&T cells). In some embodiments, an inflammatory biomarker is Glasgow Prognostic score. In some embodiments, an inflammatory biomarker is Neutrophil/Lymphocyte ratio. In some embodiments, an inflammatory biomarker is Platelet/Lymphocyte ratio Th17 lymphocytes. In some embodiments, measuring an immune-related effector level in a sample of a patient comprises using a standard clinical routine (white blood cell [(WBC]) counts). In some embodiments, measuring an immune-related effector level in a sample of a patient comprises using a flow cytometry method. In some embodiments, measuring an immune-related effector level in a sample of a patient comprises using an immunohistochemistry method. In some embodiments, an immunohistochemistry method uses a stain selected from hematoxylin and eosin. In some embodiments, measuring an immune-related effector level in a sample of a patient comprises using a multicolor flow cytometry method. In some embodiments, measuring an immune-related effector level in a sample of a patient comprises using a tissue microarray and whole tissue sections. In some embodiments, measuring an immune-related effector level in a sample of a patient comprises using a FACS method. In some embodiments, measuring an immune-related effector level in a sample of a patient comprises using Combined C-RP and albumin tests.

In some embodiments, an inflammatory biomarker is an acute phase protein. In some embodiments, an acute phase protein is a C-reactive protein. In some embodiments, an acute phase protein is Serum Amyloid A. In some embodiments, an acute phase protein is ESA. In some embodiments, measuring a C-reactive protein level in a sample of a patient comprises using an immunoassay method. In some embodiments, measuring a Serum Amyloid A level in a sample of a patient comprises using a high-sensitivity nephelometry method. In some embodiments, measuring a Serum Amyloid A level in a sample of a patient comprises using a micro-latex agglutination test. In some embodiments, measuring a C-reactive protein level in a sample of a patient comprises using a Fluorescence Polarization-immunoassay method.

In some embodiments, an inflammatory biomarker is a reactive oxygen species (ROS). In some embodiments, an inflammatory biomarker is a reactive nitrogen species (RNS). In some embodiments, an inflammatory biomarker is selected from oxidatively/nitrosatively modified DNA, or proteins. In some embodiments, an inflammatory biomarker is 3-nitrotyrosine. In some embodiments, an inflammatory biomarker is 8-hydroxy-2′-deoxyguanosine (8-oxodg or 8-OHdG). In some embodiments, an inflammatory biomarker is 8-Iso-PGF_{2_α}. In some embodiments, an inflammatory biomarker is Malondialdehyde (MDA). In some embodiments, an inflammatory biomarker is trans-4-hydroxy-2-noneal (HNE). In some embodiments, measuring a reactive oxygen species or a reactive nitrogen species level in a sample of a patient comprises measuring indirectly the product of ROS/RNS reactions. In some embodiments, measuring the product of ROS/RNS reactions comprises using an ELISA method. In some embodiments, measuring the product of ROS/RNS reactions comprises using an HPLC method. In some embodiments, measuring indirectly the product of ROS/RNS reactions comprises using a method selected from gas chromatography-mass spectrometry (GC-MS), high performance liquid chromatography coupled to electrochemical detection (HPLC-ECD), HPLC-mass spectrometry (MS), immunoassays, and enzymatic assays.

In some embodiments, an inflammatory biomarker is a prostaglandin and cyclooxygenase-related factor. In some embodiments, a prostaglandin and cyclooxygenase-related factor is selected from thromboxane, prostacyclin and prostaglandins D, E & F. In some embodiments, a prostaglandin and cyclooxygenase-related factor is COX-1 or COX-2. In some embodiments, measuring a prostaglandin and cyclooxygenase-related factor level in a sample of a patient comprises using a GC-MS method. In some embodiments, measuring a prostaglandin and cyclooxygenase-related factor level in a sample of a patient comprises using an antibody-based method, such as ELISA and RIA. In some embodiments, measuring a prostaglandin and cyclooxygenase-related factor level in a sample of a patient comprises using a LC-MS/MS method. In some embodiments, measuring a prostaglandin and cyclooxygenase-related factor level in a sample of a patient comprises using an immunohistochemistry method.

In some embodiments, an inflammatory biomarker is a transcription factor or a growth factor. In some embodiments, a transcription factor is NF-kb. In some embodiments, a transcription factor is STAT3. In some embodiments, a transcription factor is an interferon-regulatory factor IRF. In some embodiments, an interferon-regulatory factor IRF is selected from IRF1, IRF2, IRF3, IRF4, IRF5, IRF6, IRF7, IRF8, IRF9, vIRF1, vIRF2, and vIRF3. In some embodiments, measuring a transcription factor or a growth factor level in a sample of a patient comprises using an ELISA method. In some embodiments, measuring a transcription factor or a growth factor level in a sample of a patient comprises using a real-time PCR method. In some embodiments, measuring a transcription factor or a growth factor level in a sample of a patient comprises using a confocal microscopy method. In some embodiments, measuring a transcription factor or a growth factor level in a sample of a patient comprises using a flow cytometry method.

In some embodiments, an inflammatory biomarker is erythrocyte sedimentation rate (ESR). In some embodiments, an inflammatory biomarker is procalcitonin (PCT).

In some embodiments, an inflammatory biomarker is a cutaneous inflammatory biomarker. In some embodiments, an inflammatory biomarker is a circulating inflammatory biomarker. In some embodiments, an inflammatory biomarker is IRAK4 in circulating peripheral blood mononuclear cells (PBMC), for example, B cells, CD4−/CD8− (double-negative, DN) T cells, CD4+ T cells, CD8+ T cells, monocytes, and NK cells.

In some embodiments, an inflammatory biomarker is IRAK4 in B cells. In some embodiments, a patient has an elevated level of IRAK4 in B cells. In some embodiments, IRAK4 in B cells of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is IRAK4 in DN T cells. In some embodiments, a patient has an elevated level of IRAK4 in DN T cells. In some embodiments, IRAK4 in DN T cells of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is IRAK4 in CD4+ T cells. In some embodiments, a patient has an elevated level of IRAK4 in CD4+ T cells. In some embodiments, IRAK4 in CD4+ T cells of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is IRAK4 in CD8+ T cells. In some embodiments, a patient has an elevated level of IRAK4 in CD8+ T cells. In some embodiments, IRAK4 in CD8+ T cells of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is IRAK4 in monocytes. In some embodiments, a patient has an elevated level of IRAK4 in monocytes. In some embodiments, IRAK4 in monocytes of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is IRAK4 in NK cells. In some embodiments, a patient has an elevated level of IRAK4 in NK cells. In some embodiments, IRAK4 in NK cells of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is a chemokine selected from CCL2, CCL20, CSF3, CXCL1, CXCL2, CXCL6, CXCL8, CXCL11, and CXCL13. In some embodiments, a patient has an elevated level of a chemokine selected from CCL2, CCL20, CSF3, CXCL1, CXCL2, CXCL6, CXCL8, CXCL11, and CXCL13 in the skin, for example, in HS skin lesions. In some embodiments, a chemokine selected from CCL2, CCL20, CSF3, CXCL1, CXCL2, CXCL6, CXCL8, CXCL11, and CXCL13 in the skin, for example, in HS skin lesions, of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is selected from GZMB and PRF1. In some embodiments, a patient has an elevated level of GZMB and/or PRF1 in the skin, for example, in HS skin lesions. In some embodiments, an inflammatory biomarker selected from GZMB and PRF1 in the skin, for example, in HS skin lesions, of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is a cytokine selected from IFNG, IL10, IL1B, IL32, IL36G, IL6, IRF7, SOCS3, and TNF. In some embodiments, a patient has an elevated level of a cytokine selected from IFNG, IL10, IL1B, IL32, IL36G, IL6, IRF7, SOCS3, and TNF in the skin, for example, in HS skin lesions. In some embodiments, a cytokine selected from IFNG, IL10, IL1B, IL32, IL36G, IL6, IRF7, SOCS3, and TNF in the skin, for example, in HS skin lesions, of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is a cytokine receptor selected from IL2RA, IL2RB, and IL18RAP. In some embodiments, a patient has an elevated level of a cytokine receptor selected from IL2RA, IL2RB, and IL18RAP in the skin, for example, in HS skin lesions. In some embodiments, a cytokine receptor selected from IL2RA, IL2RB, and IL18RAP in the skin, for example, in HS skin lesions, of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is selected from MYD88, TLR1, TLR2, TLR3, TLR4, TLR6, TLR8, and TLR9. In some embodiments, a patient has an elevated level of MYD88, TLR1, TLR2, TLR3, TLR4, TLR6, TLR8, and/or TLR9 in the skin, for example, in HS skin lesions. In some embodiments, an inflammatory biomarker selected from MYD88, TLR1, TLR2, TLR3, TLR4, TLR6, TLR8, and TLR9 in the skin, for example, in HS skin lesions, of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is selected from NLRP3 and PTGS2. In some embodiments, a patient has an elevated level of NLRP3 and/or PTGS2 in the skin, for example, in HS skin lesions. In some embodiments, an inflammatory biomarker selected from NLRP3 and PTGS2 in the skin, for example, in HS skin lesions, of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is selected from CXCL6, CXCL8, CXCL1, CGAS, SOCS3, CXCL13, and CTSL. In some embodiments, a patient has an elevated level of CXCL6, CXCL8, CXCL1, CGAS, SOCS3, CXCL13, and/or CTSL in the skin, for example, in HS skin lesions. In some embodiments, an inflammatory biomarker selected from CXCL6, CXCL8, CXCL1, CGAS, SOCS3, CXCL13, and CTSL in the skin, for example, in HS skin lesions, of a patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

In some embodiments, an inflammatory biomarker is selected from those described in the Examples.

In some embodiments, an elevated level an inflammatory biomarker refers to a concentration or amount of an inflammatory biomarker in a sample, which is higher by about 5%, about 10%, about 15%, about 20%, about 25%, about 30%, about 35%, about 40%, about 5%, about 50%, about 55%, about 60%, about 65%, about 70%, about 75%, about 80%, about 85%, about 90%, about 95%, about 96%, about 97%, about 98%, about 99%, about 100%, about 1.5 fold, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, than the concentration or amount of an inflammatory biomarker in a control sample or control samples, such as an individual or group of individuals who are not suffering from the disease or disorder (e.g., hidradenitis suppurativa and/or atopic dermatitis), or a control sample databased based on retrospective patient sample analysis, or an internal control, as determined by techniques known in the art. In some embodiments, an elevated level an inflammatory biomarker refers to a concentration or amount of an inflammatory biomarker in a sample, which is higher by one standard deviation, two standard deviations, three standard deviations, four standard deviations, five standard deviations, or more, relative to the mean (average) or median amount or concentration of an inflammatory biomarker in a control group of samples or a baseline group of samples. In some embodiments, a mean (average) or median amount or concentration of an inflammatory biomarker in a control group of samples or a baseline group of sample is previously determined, or measured prior to the measurement in the sample, or obtained from a database of such control samples.

In some embodiments, a reduced level of an inflammatory biomarker refers to a concentration or amount of an inflammatory biomarker in a sample, which is lower by about 5%, about 10%, about 15%, about 20%, about 25%, about 30%, about 35%, about 40%, about 5%, about 50%, about 55%, about 60%, about 65%, about 70%, about 75%, about 80%, about 85%, about 90%, about 95%, about 96%, about 97%, about 98%, about 99%, about 100%, about 1.5 fold, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or more, than the concentration or amount of an inflammatory biomarker in a control sample or control samples, such as an individual or group of individuals who are not suffering from the disease or disorder (e.g., hidradenitis suppurativa and/or atopic dermatitis), or a control sample databased based on retrospective patient sample analysis, or an internal control, as determined by techniques known in the art. In some embodiments, a reduced level an inflammatory biomarker refers to a concentration or amount of an inflammatory biomarker in a sample, which is lower by one standard deviation, two standard deviations, three standard deviations, four standard deviations, five standard deviations, or more, relative to the mean (average) or median amount or concentration of an inflammatory biomarker in a control group of samples or a baseline group of samples. In some embodiments, a mean (average) or median amount or concentration of an inflammatory biomarker in a control group of samples or a baseline group of sample is previously determined, or measured prior to the measurement in the sample, or obtained from a database of such control samples.

In some embodiments, measuring a level of an inflammatory biomarker in a sample comprises normalizing the concentration or amount of an inflammatory biomarker in a sample against a control sample or control samples, such as an individual or group of individuals who are not suffering from the disease or disorder (e.g., hidradenitis suppurativa and/or atopic dermatitis), or a control sample databased based on retrospective patient sample analysis, or an internal control, as determined by techniques known in the art. In some embodiments, an elevated level of an inflammatory biomarker refers to a concentration or amount of an inflammatory biomarker normalized to the concentration or amount of a control sample or control samples, which is higher by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 1.5 fold, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, than a normal normalized concentration or amount of an inflammatory biomarker in a sample, or a selected or prespecified or predefined normalized amount or concentration of an inflammatory biomarker in a sample. In some embodiments, an elevated level of an inflammatory biomarker refers to a concentration or amount of an inflammatory biomarker in a sample normalized to the concentration or amount of a control sample or control samples, which is higher by one standard deviation, two standard deviations, three standard deviations, four standard deviations, five standard deviations, or more, relative to the mean (average) or median amount or concentration of an inflammatory biomarker in a control group of samples or a baseline group of samples. In some embodiments, a mean (average) or median amount or concentration of an inflammatory biomarker in a control group of samples or a baseline group of sample is previously determined, or measured prior to the measurement in the sample, or obtained from a database of such control samples.

In some embodiments, a reduced level of an inflammatory biomarker refers to a concentration or amount of an inflammatory biomarker normalized to the concentration or amount of a control sample or control samples, which is lower by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 1.5 fold, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or more, than a normal normalized concentration or amount of an inflammatory biomarker in a sample, or a selected or prespecified or predefined normalized amount or concentration of an inflammatory biomarker in a sample. In some embodiments, a reduced level of an inflammatory biomarker refers to a concentration or amount of an inflammatory biomarker in a sample normalized to the concentration or amount of a control sample or control samples, which is lower by one standard deviation, two standard deviations, three standard deviations, four standard deviations, five standard deviations, or more, relative to the mean (average) or median amount or concentration of an inflammatory biomarker in a control group of samples or a baseline group of samples. In some embodiments, a mean (average) or median amount or concentration of an inflammatory biomarker in a control group of samples or a baseline group of sample is previously determined, or measured prior to the measurement in the sample, or obtained from a database of such control samples.

In another aspect, the present invention provides a method of treating a disease or disorder in a patient having an elevated level of an inflammatory biomarker, comprising administering to the patient a therapeutically effective amount of an IRAK degrader.

In some embodiments, the present invention provides a method of treating a disease or disorder in a patient, comprising selecting a patient having an elevated level of an inflammatory biomarker, and administering to the patient a therapeutically effective amount of an IRAK degrader.

In some embodiments, the present invention provides a method of treating a disease or disorder in a patient, comprising measuring an inflammatory biomarker level in a sample of a patient, selecting a patient having an elevated level of an inflammatory biomarker, and administering to the patient a therapeutically effective amount of an IRAK degrader.

In another aspect, the present invention provides a method of treating a disease or disorder in a patient having a reduced level of an inflammatory biomarker after a treatment with an IRAK degrader, comprising administering to the patient a therapeutically effective amount of an IRAK degrader.

In some embodiments, the present invention provides a method of treating a disease or disorder in a patient, comprising selecting a patient having a reduced level of an inflammatory biomarker after a treatment with an IRAK degrader, and administering to the patient a therapeutically effective amount of an IRAK degrader.

In some embodiments, the present invention provides a method of treating a disease or disorder in a patient, comprising measuring an inflammatory biomarker level in a sample of a patient after a treatment with an IRAK degrader, selecting a patient having a reduced level of an inflammatory biomarker after a treatment with an IRAK degrader, and administering to the patient a therapeutically effective amount of an IRAK degrader.

In some embodiments, the present invention provides a method of treating a disease or disorder in a patient, comprising administering to a patient a therapeutically effective amount of an IRAK degrader, measuring an inflammatory biomarker level in a sample of a patient after a treatment with an IRAK degrader, selecting a patient having a reduced level of an inflammatory biomarker after a treatment with an IRAK degrader, and administering to the patient a therapeutically effective amount of an IRAK degrader.

In some embodiments, a disease or disorder is hidradenitis suppurativa. In some embodiments, a disease or disorder is active mild, moderate, or severe hidradenitis suppurativa. In some embodiments, active mild, moderate, or severe hidradenitis suppurativa is determined by an HS-PGA assessment. In some embodiments, a disease or disorder is an atopic dermatitis. In some embodiments, a disease or disorder is active moderate or severe atopic dermatitis. In some embodiments, active moderate or severe atopic dermatitis is determined by a PGA assessment.

In some embodiments, a treatment method provided herein is to treat a patient, who is not, or has not been, on a biologic or other immunosuppressive treatment for HS or AD. In some embodiments, a treatment method provided herein is to treat a patient, who is not, or has not been, on a biologic treatment for HS or AD within 3 months or 5 half-lives, whichever is longer. In some embodiments, a treatment method provided herein is to treat a patient, who is not, or has not been, on a non-biologic immunosuppressive treatment (eg. Cyclosporin) within 4 weeks.

In some embodiments, an IRAK degrader is an IRAK1 degrader. In some embodiments, an IRAK degrader is an IRAK2 degrader. In some embodiments, an IRAK degrader is an IRAK3 degrader. In some embodiments, an IRAK degrader is an IRAK4 degrader. In some embodiments, an IRAK degrader is selected from those described in WO 2019/133531 and WO 2020/010227, the contents of each of which are incorporated herein by reference in their entireties.

### 4. Formulation and Administration

In some embodiments, a method described herein comprises administering a pharmaceutical composition comprising an IRAK degrader, as described herein, and a pharmaceutically acceptable carrier, adjuvant, or vehicle. In some embodiments, the amount of an IRAK degrader in a composition is such that is effective to measurably decrease the activity of IRAK, including IRAK1, IRAK2, IRAK3, and/or IRAK4, in a biological sample or in a patient. In some embodiments, an IRAK degrader composition is formulated for oral administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. In some embodiments, provided compositions are formulated so that a dosage of between 0.01-100 mg/kg body weight/day of an IRAK degrader can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Exemplary Embodiments

Embodiment 1. A method of measuring an inflammatory biomarker level in a patient, comprising measuring an inflammatory biomarker level in a sample of the patient.

Embodiment 2. A method of identifying or selecting a patient having an elevated level of an inflammatory biomarker, comprising measuring a level of an inflammatory biomarker in a sample of a patient, and selecting a patient having an elevated level of an inflammatory biomarker in a sample.

Embodiment 3. A method of identifying or selecting a patient having a reduced level of an inflammatory biomarker after a treatment with an IRAK degrader, comprising measuring a level of an inflammatory biomarker in a sample of a patient after a treatment with an IRAK degrader, and selecting a patient having a reduced level of an inflammatory biomarker in a sample.

Embodiment 4. A method of identifying or selecting a patient having a reduced level of an inflammatory biomarker after a treatment with an IRAK degrader, comprising administering to a patient a therapeutically effective amount of an IRAK degrader, measuring a level of an inflammatory biomarker in a sample of a patient after a treatment with an IRAK degrader, and selecting a patient having a reduced level of an inflammatory biomarker in a sample after a treatment with an IRAK degrader.

Embodiment 5. The method of any one of Embodiments 1-4, wherein the patient is a hidradenitis suppurativa patient, and/or an atopic dermatitis patient.

Embodiment 6. The method of any one of Embodiments 1-5, wherein the sample is a blood sample or a skin sample.

Embodiment 7. The method of any one of Embodiments 1-6, wherein the inflammatory biomarker is a cytokine.

Embodiment 8. The method of any one of Embodiments 1-6, wherein the inflammatory biomarker is an immune-related effector.

Embodiment 9. The method of any one of Embodiments 1-6, wherein the inflammatory biomarker is an acute phase protein.

Embodiment 10. The method of any one of Embodiments 1-6, wherein the inflammatory biomarker is a reactive oxygen species (ROS) or a reactive nitrogen species (RNS).

Embodiment 11. The method of any one of Embodiments 1-6, wherein the inflammatory biomarker is a prostaglandin and cyclooxygenase-related factor.

Embodiment 12. The method of any one of Embodiments 1-6, wherein the inflammatory biomarker is a transcription factor or a growth factor.

Embodiment 13. The method of any one of Embodiments 1-6, wherein the inflammatory biomarker is erythrocyte sedimentation rate (ESR) or procalcitonin (PCT).

Embodiment 14. A method of treating a disease or disorder in a patient having an elevated level of an inflammatory biomarker, comprising administering to the patient a therapeutically effective amount of an IRAK degrader.

Embodiment 15. A method of treating a disease or disorder in a patient, comprising selecting a patient having an elevated level of an inflammatory biomarker, and administering to the patient a therapeutically effective amount of an IRAK degrader.

Embodiment 16. A method of treating a disease or disorder in a patient, comprising measuring an inflammatory biomarker level in a sample of a patient, selecting a patient having an elevated level of an inflammatory biomarker, and administering to the patient a therapeutically effective amount of an IRAK degrader.

Embodiment 17. A method of treating a disease or disorder in a patient having a reduced level of an inflammatory biomarker after a treatment with an IRAK degrader, comprising administering to the patient a therapeutically effective amount of an IRAK degrader.

Embodiment 18. A method of treating a disease or disorder in a patient, comprising selecting a patient having a reduced level of an inflammatory biomarker after a treatment with an IRAK degrader, and administering to the patient a therapeutically effective amount of an IRAK degrader.

Embodiment 19. A method of treating a disease or disorder in a patient, comprising measuring an inflammatory biomarker level in a sample of a patient after a treatment with an IRAK degrader, selecting a patient having a reduced level of an inflammatory biomarker after a treatment with an IRAK degrader, and administering to the patient a therapeutically effective amount of an IRAK degrader.

Embodiment 20. A method of treating a disease or disorder in a patient, comprising administering to a patient a therapeutically effective amount of an IRAK degrader, measuring an inflammatory biomarker level in a sample of a patient after a treatment with an IRAK degrader, selecting a patient having a reduced level of an inflammatory biomarker after a treatment with an IRAK degrader, and administering to the patient a therapeutically effective amount of an IRAK degrader.

Embodiment 21. The method of any one of Embodiments 14-20, wherein the disease or disorder is hidradenitis suppurativa patient and/or atopic dermatitis.

Embodiment 22. The method of any one of Embodiments 16, 19, or 20, wherein the sample is a blood sample or a skin sample.

Embodiment 23. The method of any one of Embodiments 14-22, wherein the inflammatory biomarker is a cytokine.

Embodiment 24. The method of any one of Embodiments 14-22, wherein the inflammatory biomarker is an immune-related effector.

Embodiment 25. The method of any one of Embodiments 14-22, wherein the inflammatory biomarker is an acute phase protein.

Embodiment 26. The method of any one of Embodiments 14-22, wherein the inflammatory biomarker is a reactive oxygen species (ROS) or a reactive nitrogen species (RNS).

Embodiment 27. The method of any one of Embodiments 14-22, wherein the inflammatory biomarker is a prostaglandin and cyclooxygenase-related factor.

Embodiment 28. The method of any one of Embodiments 14-22, wherein the inflammatory biomarker is a transcription factor or a growth factor.

Embodiment 29. The method of any one of Embodiments 14-22, wherein the inflammatory biomarker is erythrocyte sedimentation rate (ESR) or procalcitonin (PCT).

### EXEMPLIFICATION

The following examples are provided for illustrative purposes only and are not to be construed as limiting this invention in any manner.

### Abbreviation Definition

### AD Atopic Dermatitis

### BSA Body Surface Area

### EAST Eczema Area Severity Index

### GISS Global Individual Signs Score

### HS Hidradenitis Suppurativa

### IHS4 International HS Severity Scoring System

### IRAK Interleukin 1 Receptor Associated Kinase 4

### IL Interleukin

### MYD88 Myeloid Differentiation Primary Response 88

### PGA Physician Global Assessment

### PI Principle Investigator

### RNA Ribonucleic Acid

### TCS Topical Corticosteroid

Th2 T helper cell type 2
TLR Toll-like receptor

### Example 1. Non-Interventional Study to Evaluate Cutaneous and Circulating Inflammatory Biomarkers for a Novel IRAK4-Targeted Therapeutic in Hidradenitis Suppurativa and Atopic Dermatitis Patient Samples

### 1. Study Objectives

Identify the biomarker profiles in HS and AD (as a comparator) that would have the most utility in interventional studies evaluating efficacy during therapeutic intervention. Correlate cellular/molecular changes in blood (cells and serum), and tissue, with clinical/histopathological phenotypes. Assess ex-vivo treatment effects on IRAK4 levels and inflammatory markers in blood.

### 2. Number of Subjects:

Up to 30 patients with HS in different stages of mild, moderate to severe, preferably at least 10 in each subgroup, will be enrolled in the study. The study will also enroll up-to 10 patients with AD with an equal distribution of moderate and severe.

### 3. Study Design

This proposed pilot study is an exploratory correlative study in subjects with HS or AD. All subjects will have blood work, and skin biopsies to address the following 4 aims:
Aim 1: Assess cutaneous and circulating inflammatory biomarkers and IRAK4 target levels in primary samples
Aim 2: Determine correlations between cutaneous and circulating inflammatory biomarkers and between these biomarkers and disease severity
Aim 3: Examine effect of IRAK4 degrader on IRAK4 levels and downstream inflammatory biomarkers in ex vivo-treated whole blood from patients De-identified routine clinical data that is collected will be correlated with the research findings.
   Patient data
      a. Age, gender, race and ethnicity
   Patient and Family history
      a. Concomitant treatments (prior and current), co-morbidities.
      b. Duration of disease (time since HS or AD diagnosis)
      c. Physical Exam
   Clinical examination for HS patients
      a. Record should be made of the site and side of involvement and the type of lesions seen. Most common lesions are nodules, abscesses, tunnels (also called tracts, sinuses, fistulae), and scars. Hurley staging at each site, HS-PGA and IHS4 can be recorded.
      b. The number of nodules, abscesses, and tunnels should be noted in each anatomic site involved.
      c. Presence (Y) or absence (N) of ulceration and scars should be noted. Diameter of lesions can be recorded.
      d. The other types of lesions that are often seen in HS can be noted.
      e. For large plaque-like lesions (often on thighs and buttocks) percent of BSA can be recorded (palm represents 1% BSA).
   Clinical examination for AD patients
      a. Extent of disease will be measured using the EASI, and BSA.
      b. Severity will be graded using PGA and GISS
   Photography
      a. If the patient consents, photographs of the areas of involvement and biopsy sites will be taken.

### 4. Study Population

### 4.1 Inclusion

1. Age 18 or older
2. Active HS or AD disease, diagnosed by PI
3. Patients with mild, moderate, or severe disease using the HS-PGA or PGA assessment.
4. Must sign an informed consent form (ICF) indicating that he or she understands the purpose of and procedures required for the study, and is willing to participate in the study.

### 4.2 Exclusion Criteria

1. Patients is currently on a biologic or other immunosuppressive treatment for HS or AD.
   2. Use of biologic treatment for HS or AD within 3 months or 5 half-lives, whichever is longer
3. Use of non-biologic immunosuppressive treatment (eg. Cyclosporin) in the last 4 weeks. Note: Antibiotics use is NOT an exclusion criterion

### 5. Schedule of Assessments

The following assessments will be performed at the study visit:

Informed Consent
Inclusion/Exclusion Criteria
Patient Demographics
Medical History
Lesion Count
Hurley Staging (HS patients only)
IHS4 (HS patients only)
HS-PGA or PGA assessment
GISS (AD patients only)
Photography (optional)
Plasma Biomarkers
PAXgene RNA collection
Biopsy Collection
Blood collection for ex-vivo compound treatment

### 6. Study Procedures

Up to 30 HS patients will be included, with approximately 10 in each severity stage (mild, moderate and severe). The study will also enroll up-to 10 AD patients with an equal distribution in each stage. For each aim, the patients will be studied for blood and skin at a single timepoint.

Blood will be collected into four (6 mL) sodium heparin tubes. One tube will be centrifuged and the plasma will be aliquoted into 2 tubes. Three tubes will have IRAK4 degrader, IRAK4 kinase inhibitor or DMSO control added directly to the blood.

Blood will be collected into one (2.5 mL) PAXgene RNA Tube.

A lesional, perilesional, and a non-lesional skin biopsy will be collected. Each sample will be bisected through the dermis, epidermis and subcutaneous fat. One half will be placed in a 10% neutral buffered formalin, incubated overnight and then processed to FFPE blocks using standard institutional protocol.
Lesional: palpable inflammatory lesion (Nodule or draining tunnel) for HS patients. Site of active disease for AD patients.
Perilesional: within 1-2 cm of lesional biopsy
Non-lesional: clinically unaffected skin at least 3 cm from affected area in same anatomical region. If not possible to collect from same region, contralateral unaffected area can be used.

Photographic documentation will be conducted pre-biopsy to aid in clinic-pathological correlations.

### 7. Preliminary Results

### 7.1 Patients

30 HS: 9 mild, 10 moderate, 11 Severe
2 AD

### 7.2 Demographics

Age 19-56 yrs
9 male, 23 Female
Duration of disease 1-38 years
Race: 97% were non-Hispanic or Latino
Prior treatments: antibiotics+up to 7 other therapeutics

### 7.3 Samples Collected

Whole blood, plasma, skin (lesional, peri-lesional, non-lesional)

### 7.4 Sample Analysis (Translation Assays)

IRAK4 FLOW PD in ex vivo treated whole blood
Targeted MS of IRAK4 in skin biopsies
IRAK4 immunofluorescence in skin biopsies, with nuclear stain (DAPI)
Cytokines from ex vivo treated whole blood
Plasma cytokines and acute phase reactants
Cytokines in skin biopsies

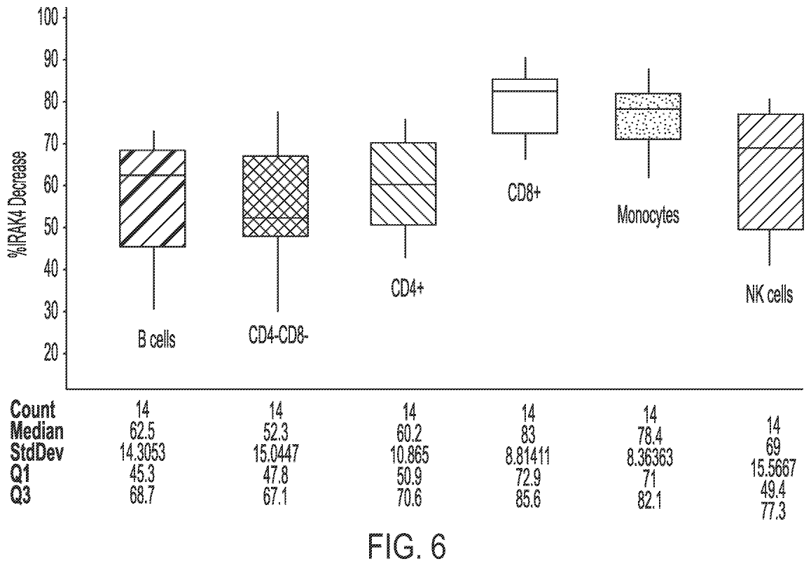

FIG. 6 shows HS patient responses to Degrader 2 ex vivo in PBMC subsets. Degrader 2 leads to IRAK degradation across multiple immune cell types.

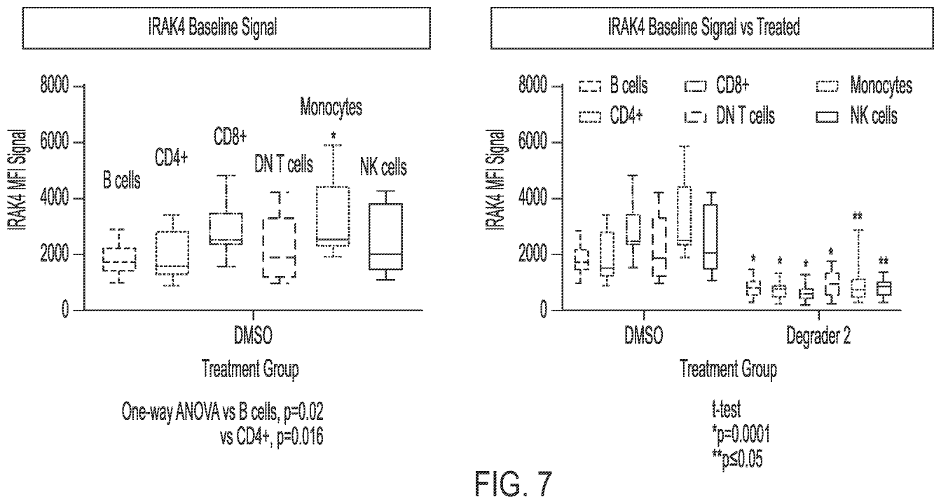

FIG. 7 shows IRAK signal at baseline and following ex vivo treatment with Degrader 2 in HS patients (n=14). The only significant difference at baseline was between monocytes versus B cells or versus CD4+ T cells. Following treatment, significant IRAK4 decreases for Degrader 2 versus DMSO was observed across all immune cell subsets. There were no significant differences between immune cell subsets in IRAK4 levels post-Degrader 2 treatment.

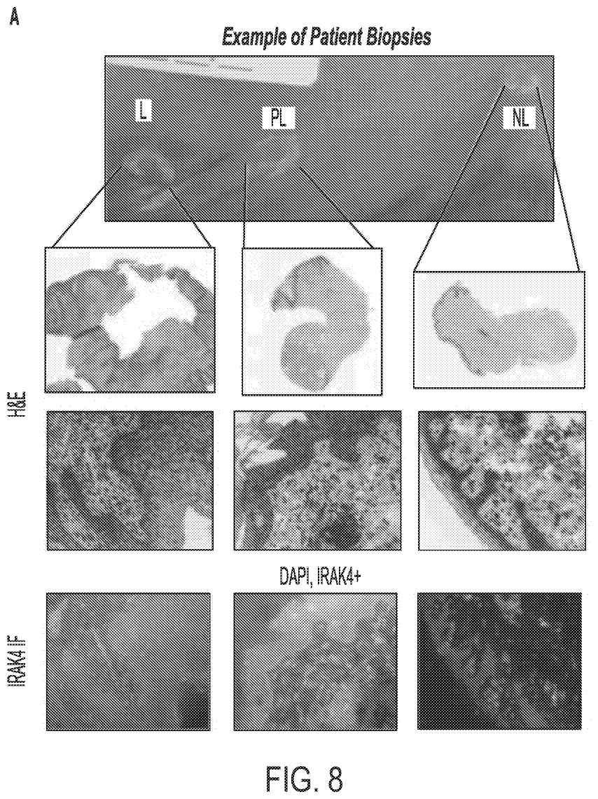

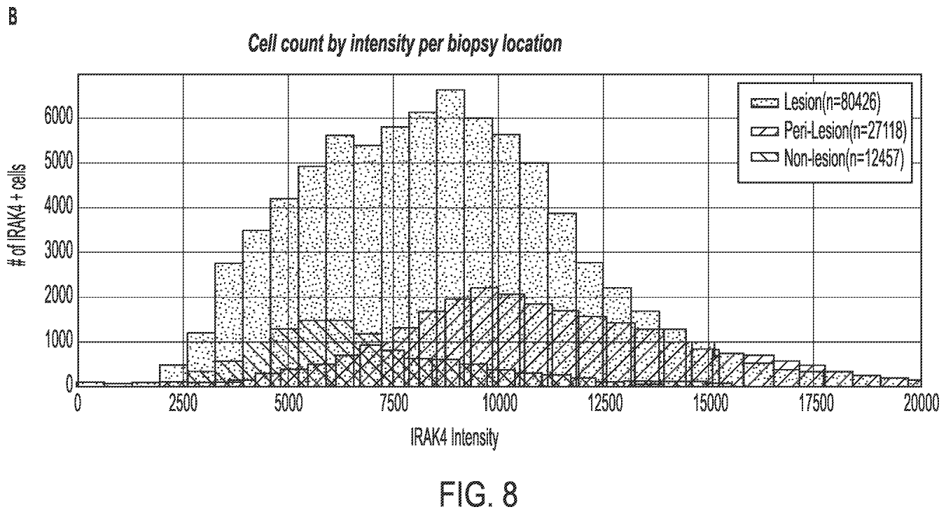

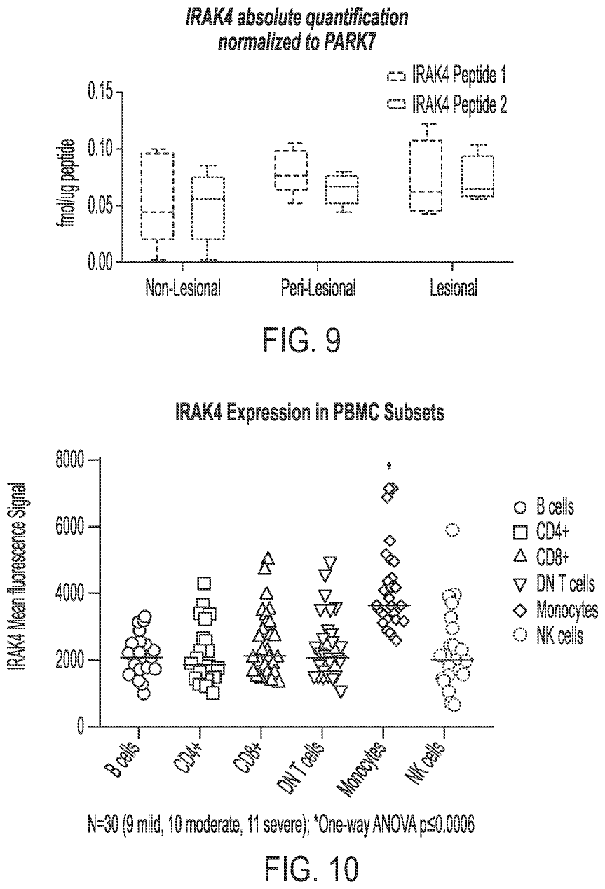

FIG. 8 shows IRAK4 immunofluorescence (IF) of patient biopsies (A) and cell count by intensity per biopsy location (B). Lesional (L), peri-lesional (PL), and non-lesional (NL) IRAK4 positive cells were counted and binned into intensity ranges as depicted by the horizontal bars in FIG. 8B. Cell counts per intensity bin were summed from the 3 biopsy locations. Two peptides were chosen providing strong concordance in absolute quantification. FIG. 9 shows absolute quantification of IRAK4 by mass spectrometry (MS) in patient biopsies normalized to PARK7. The plot represents the range of fmol/μg peptide across the 3 biopsy locations. In summary, IRAK4 expression in the skin detected by IF and MS is higher in lesional (L) and peri-lesional (PL) skin compared to non-lesional (NL) skin, supporting the relevance of the IRAK4 signaling pathway in HS.

FIG. 10 shows IRAK4 expression in peripheral blood mononuclear cells is highest in monocytes, a cell type central to the pathogenesis of HS.

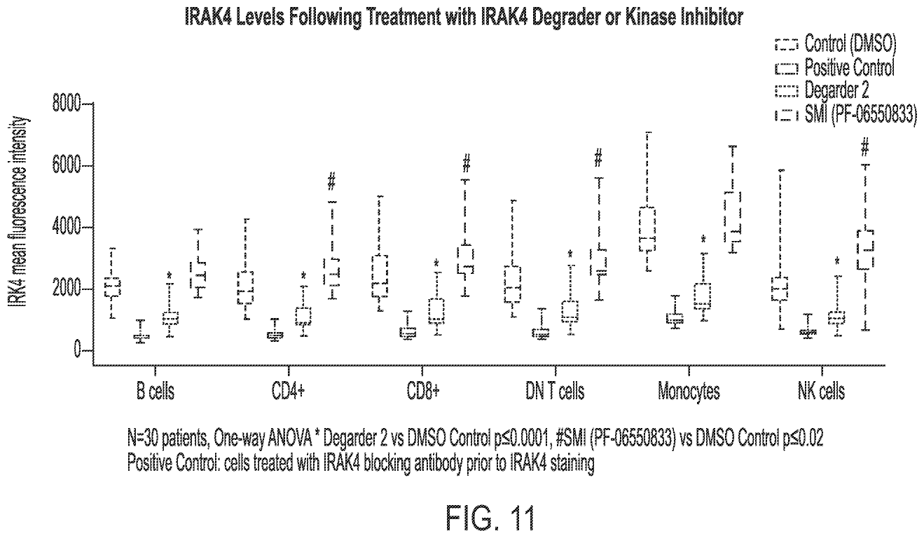

FIG. 11 shows IRAK4 degrader downregulates IRAK4 expression across all PBMC subsets with comparison to IRAK4 inhibitor. Patient blood was treated with Control DMSO or 200 nM Degrader 2 or 200 nM or small molecule inhibitor (SMI; PF-06550833). Blood was incubated overnight at 37° C. (16-24 hrs). Blood was shipped and processed for IRAK4 and lineage specific cell surfaces staining by flow cytometry. Treatment with Degrader 2 led to reduction of IRAK4 to a similar level approaching the lower limits of detection as determined by an anti-IRAK4 blocking antibody (Positive Control) across all PBMC subsets in HS patient blood, irrespective of baseline IRAK4 expression intensity. Treatment with an IRAK4 kinase inhibitor PF-06550833 led to an increase in IRAK4 levels of up to 2.6-fold in T and NK cells.

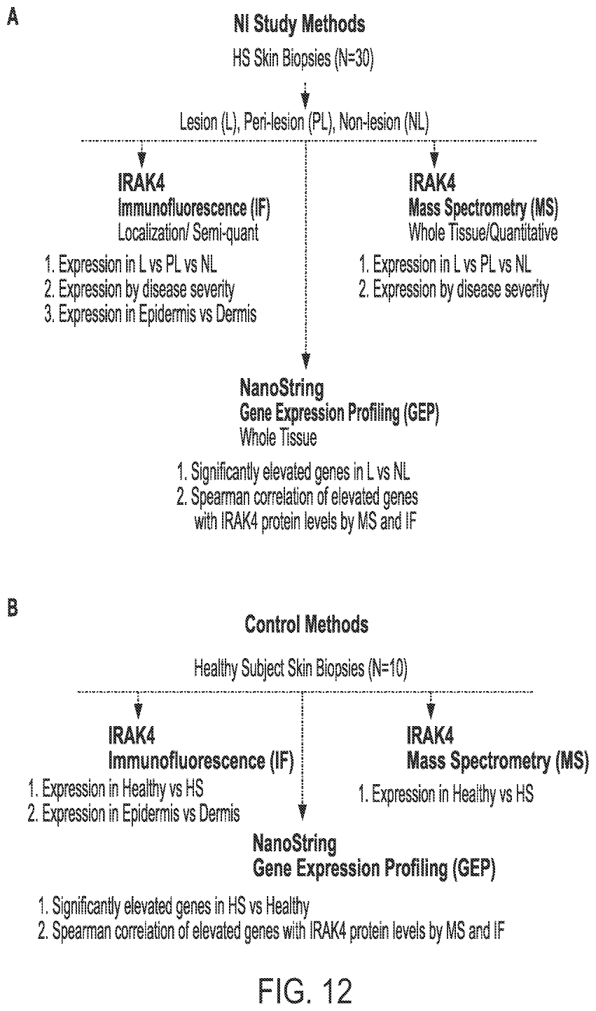

FIG. 12 shows methods for measuring IRAK4 protein and pro-inflammatory gene transcripts in HS skin biopsies (A) and healthy subject skin/monocytes (B). Ex-vivo R848-stimulated monocyte methods: 1) Mechanistic study designed to evaluate impact of IRAK4 degradation on response of healthy monocytes to TLR7/8 agonist R848; 2) Monocytes isolated from blood of healthy donors (N=3), treated overnight with 500 nM of IRAK4 Degrader 2, and then stimulated with R848; 3) For RNA-seq, cells were collected at 2 hours following stimulation; and 4) Analysis of IRAK4 Degrader 2 effect on R848 upregulation of subset of genes overexpressed in HS skin lesions that correlate with IRAK4 protein levels.

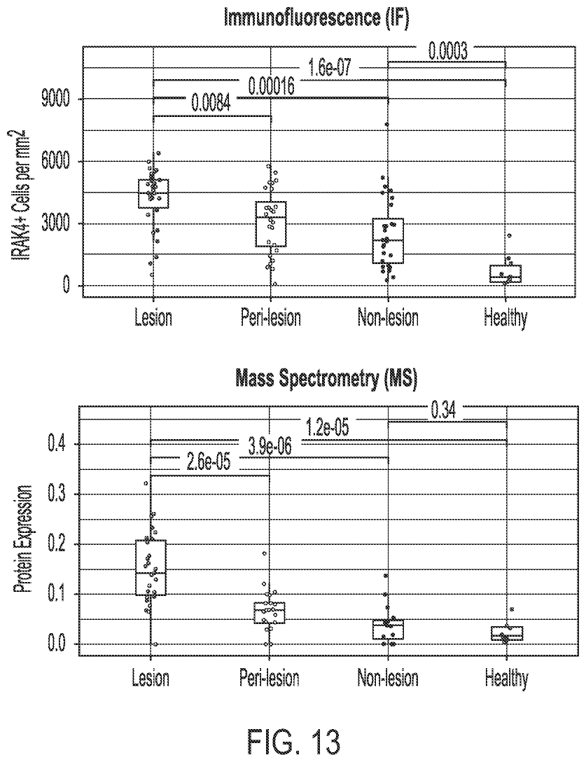

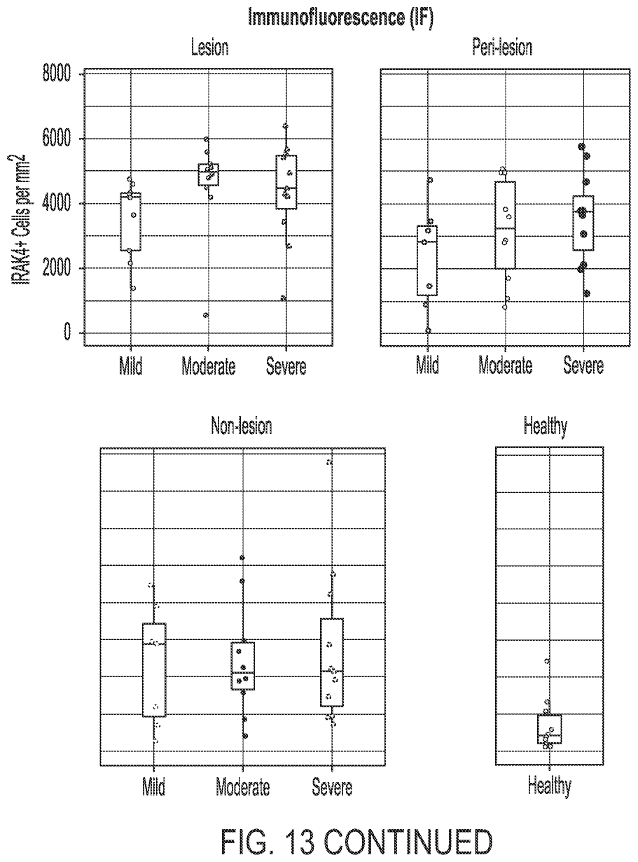

FIG. 13 shows IRAK4 protein expression is elevated in HS skin compared to skin from healthy subjects. Concordance between IF and MS for HS patients was observed. The level of IRAK4 protein expression in HS patients is Lesion>Peri-lesion>Non-lesion. IF shows significant difference between HS non-lesion skin and healthy subject skin.

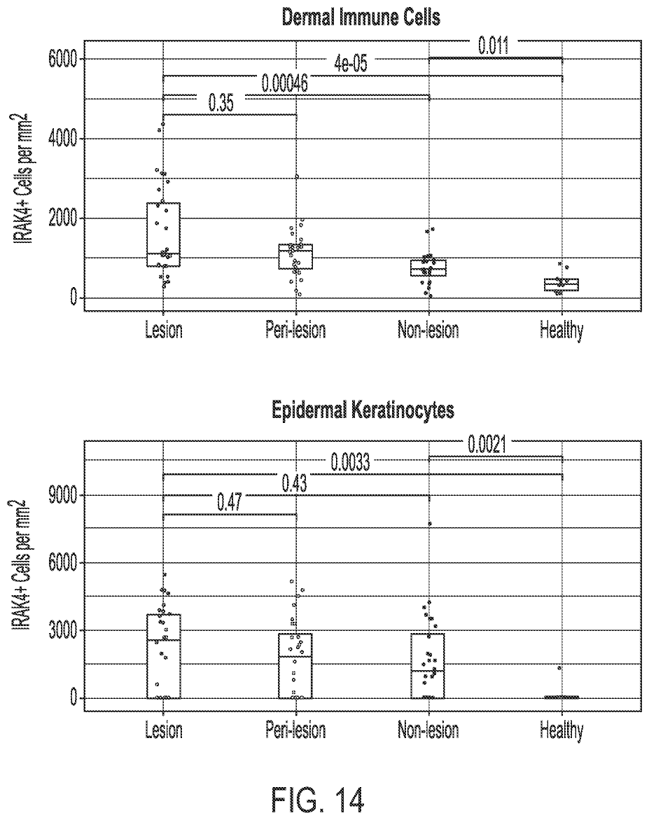

FIG. 14 shows IRAK4 is upregulated in dermis and epidermis of HS patients relative to skin of healthy subjects. IF shows increased number of IRAK4+ immune cells in dermis with HS Lesion/Peri-lesion>HS Non-lesion>Healthy subjects. Epidermal IRAK4 positivity is similar across biopsy sites in HS patients but significantly higher compared to Healthy subjects.

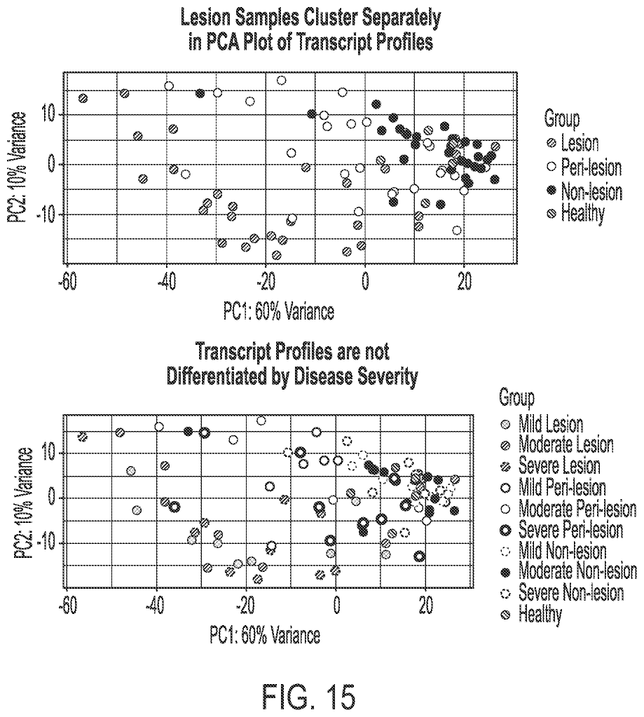

FIG. 15 depicts transcriptional profiling which shows clear differences between HS skin biopsy sites, but not across disease severity. Lesion samples show many upregulated genes relative to Peri- and Non-lesion samples.

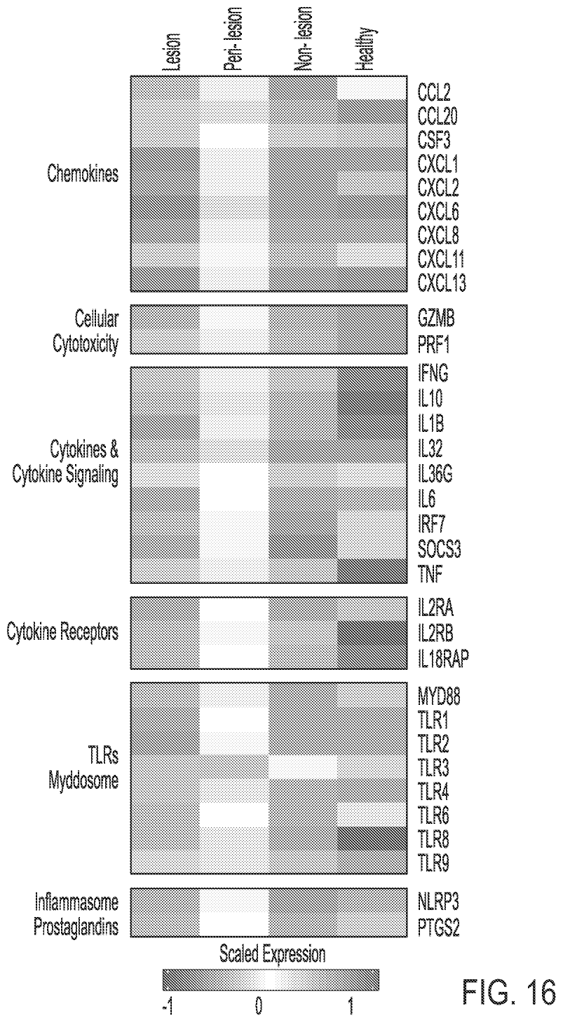

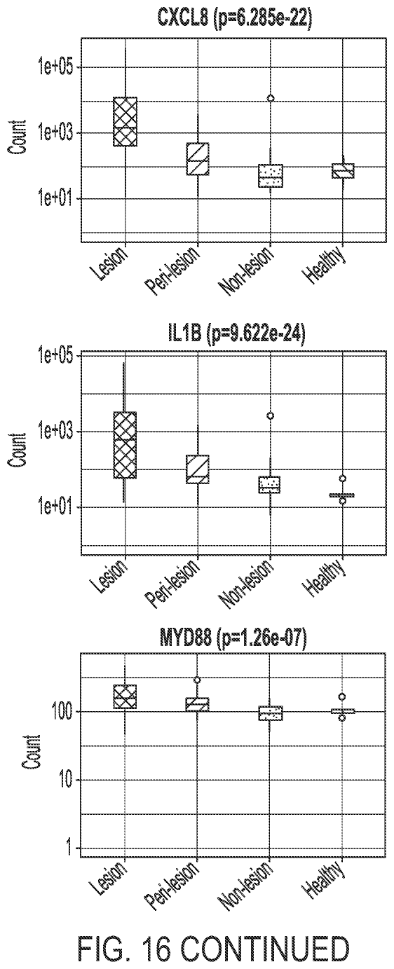

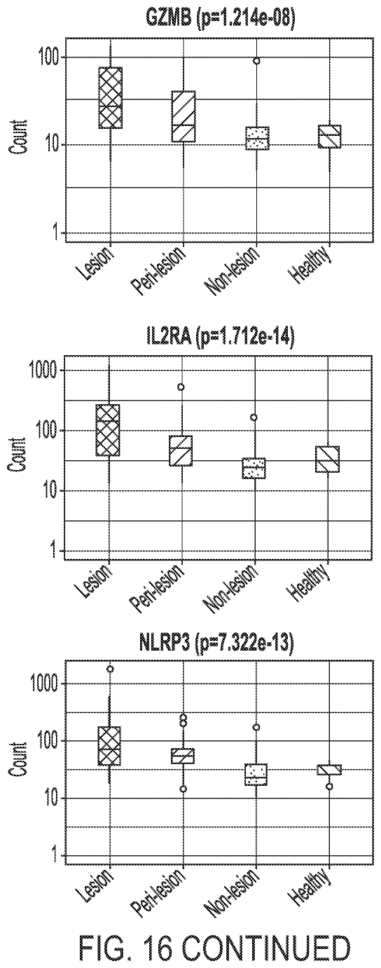

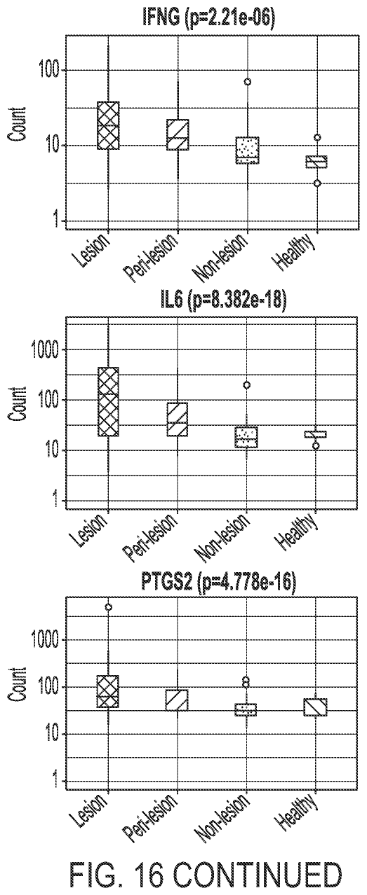

FIG. 16 shows transcripts for multiple mediators of inflammation are upregulated in HS skin lesions.

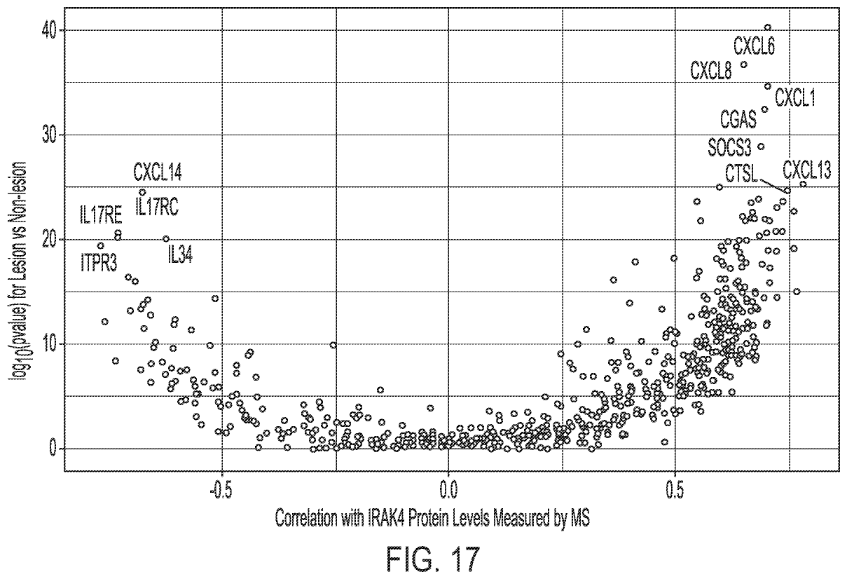

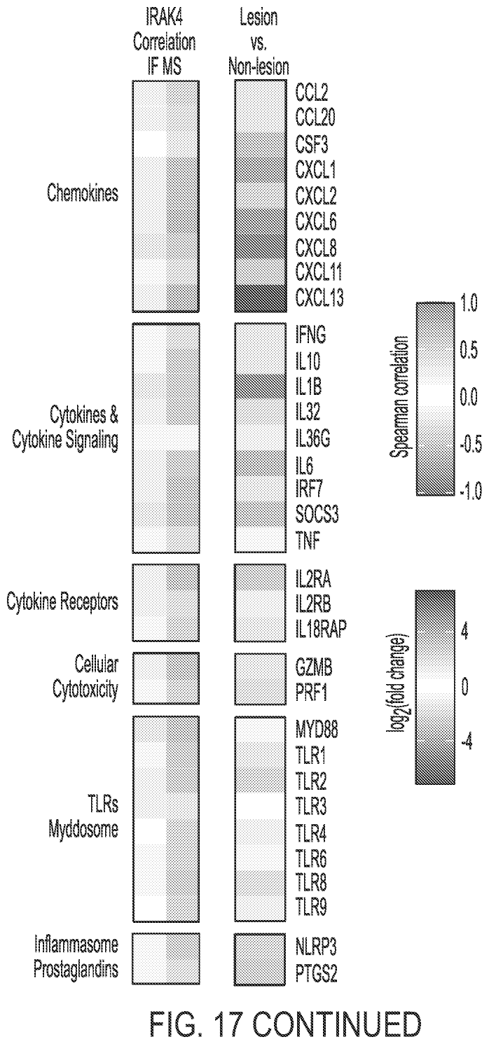

FIG. 17 shows multiple proinflammatory transcripts correlate with IRAK4 protein levels in HS skin lesions.

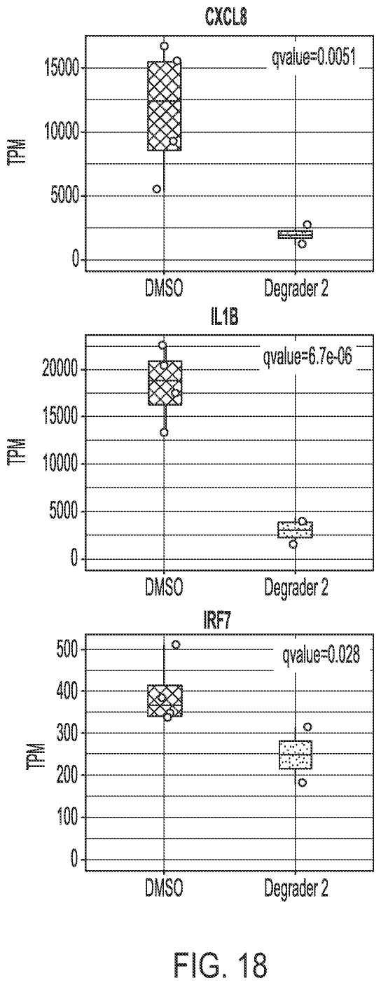

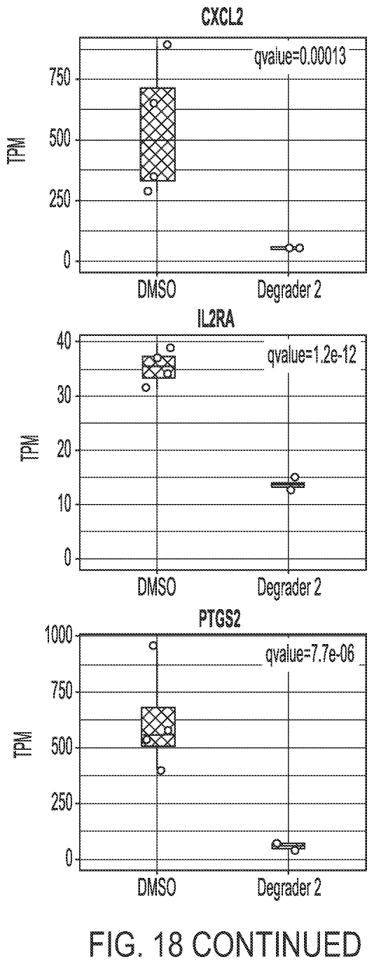

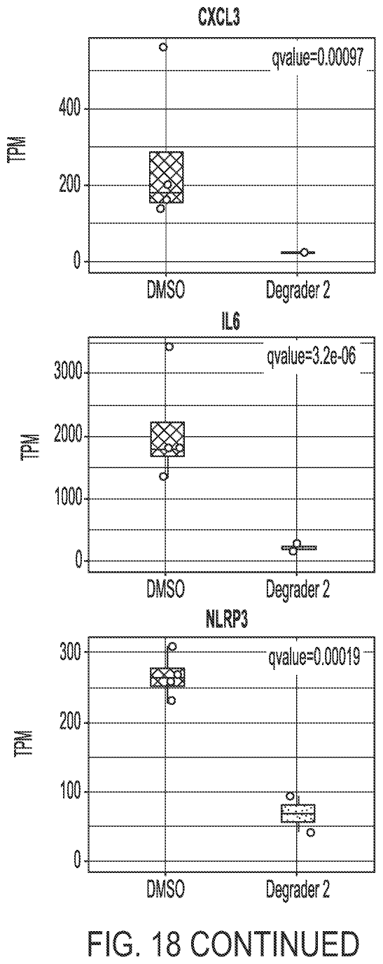

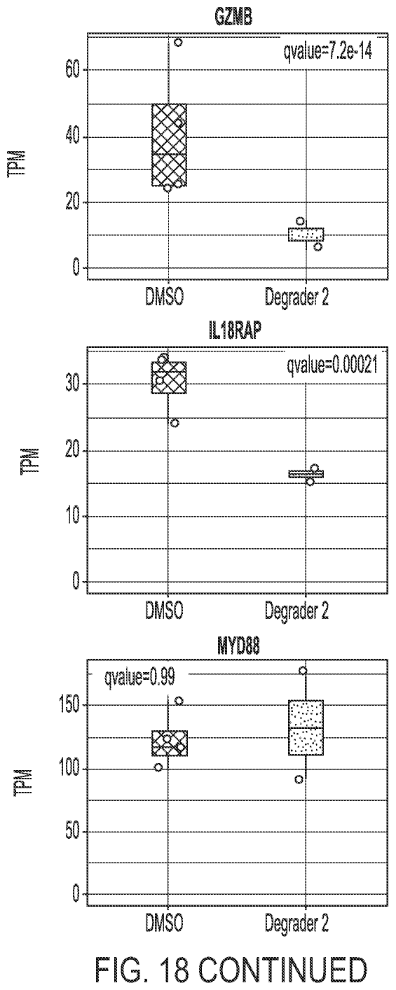

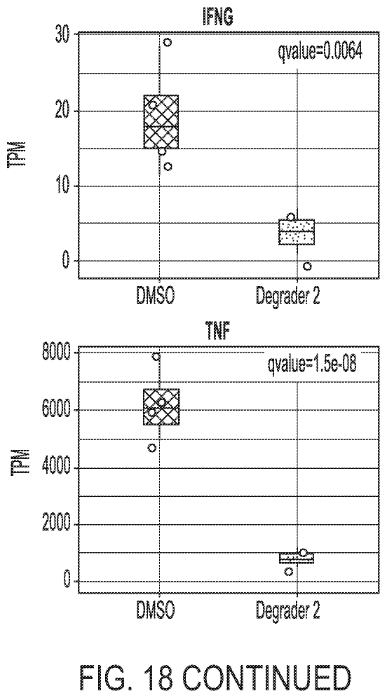

FIG. 18 shows IRAK4 Degrader 2 inhibits TLR-mediated induction of HS-overexpressed proinflammatory transcripts in healthy monocytes.

### CONCLUSIONS

IRAK4 is overexpressed in HS skin relative to healthy subjects due to increase in number of IRAK4+ dermal immune cells and epidermal keratinocytes. Higher expression in active HS skin Lesions compared to Peri-lesion and/or Non-lesion skin associated with increase in infiltrating IRAK4+ dermal immune cells. Higher expression in dermis and epidermis of Non-lesion skin compared to skin of Healthy subjects raises possibility that IRAK4 overexpression may predispose to inflammatory lesion formation in HS.

Gene expression profiling shows upregulation of multiple mediators of inflammation in HS skin lesions that correlates with IRAK4 protein overexpression, including genes involved in TLR/myddosome signaling, inflammasome activity, prostaglandin generation, Th1 and Th17 inflammation, and monocyte/neutrophil migration and activation, thereby linking IRAK4 to the pleiotropic inflammation in HS. Neither proinflammatory gene expression nor IRAK4 protein expression correlated with disease severity, suggesting common pathophysiology underlying inflammation in active lesions irrespective of disease stage.

IRAK4 Degrader 2 inhibits TLR-stimulated upregulation of HS-overexpressed inflammatory genes in monocytes from healthy subjects. This provides further evidence for role of IRAK4 in overexpression of these mediators of inflammation in HS skin Lesions and rationale for targeting IRAK4 with IRAK4 Degraders for the treatment of patients with HS.

### Example 2. Identification of Highly Potent and Selective Interleukin-1 Receptor Associated Kinase 4 (IRAK4) Degraders for the Treatment of Hidradenitis Suppurativa

Interleukin-1 receptor associated kinase 4 (IRAK4) plays a central role in myddosome signaling via kinase and scaffolding functions, making it an attractive target for the treatment of TLR- and IL-1R-driven inflammatory diseases. IL-1 family cytokines and TLRs, are central to the pathophysiology of hidradenitis suppurativa (HS), a Th1- and Th17-mediated neutrophilic, chronic inflammatory skin disease. Orally administered hetero-bifunctional molecules have been developed that selectively target IRAK4 for degradation and elimination by the ubiquitin proteasome pathway. These degraders have broad and potent activity in vitro against IL-6, TNF-α and other proinflammatory cytokines and chemokines induced by TLR agonists and IL-1 family cytokines that is superior to IRAK4 kinase inhibitors. The ability to strongly suppress inflammation and superiority over small molecule kinase inhibitors is even more pronounced after combination of TLR agonists and IL-1β. In vivo, orally-dosed IRAK4 degraders are well-tolerated in rodent and dog species and achieve exposures leading to >95% protein knockdown in spleen, PBMC and skin. IRAK4 degraders are highly active in the mouse imiquimod psoriasis model, with reduction of skin thickening and both Th1 and Th17 cytokines. Additionally, IRAK4 degraders block neutrophil infiltration and IL-1b production in the mouse MSU air-pouch model. The demonstrated activity against TLR- and IL-1R-driven Th1 and Th17 inflammation in vitro and in vivo, coupled with favorable drug-like properties and strong pharmacodynamic effect in both circulating immune cells and skin, supports the development of IRAK4 degraders in HS and other autoimmune diseases.

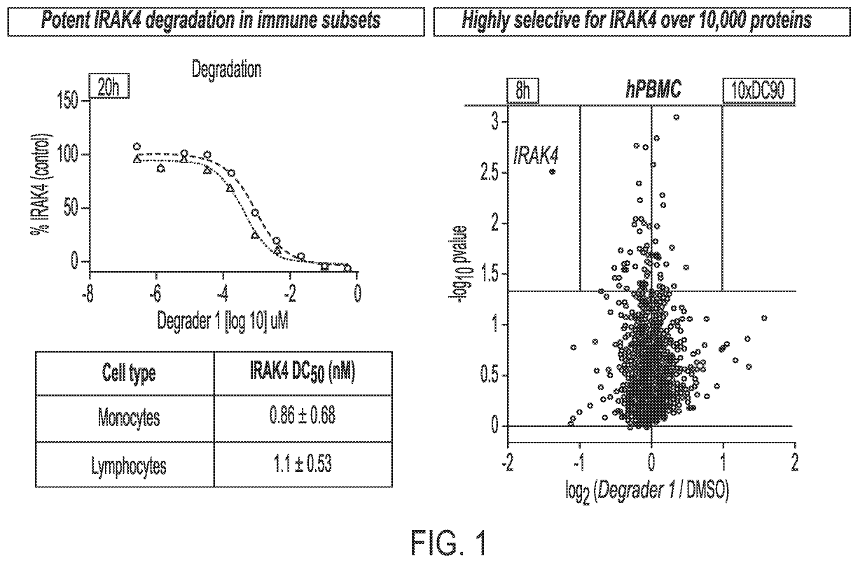

PBMC cells were treated with Degrader 1 at indicated times (20 hours or 8 hours). IRAK4 degradation was detected by flow cytometry methods and concentration where 50% degradation achieved is reported as DC50. Selectivity was assessed by Mass tandem deep proteomics with a depth of over 10,000 proteins. The results are shown in FIG. 1.

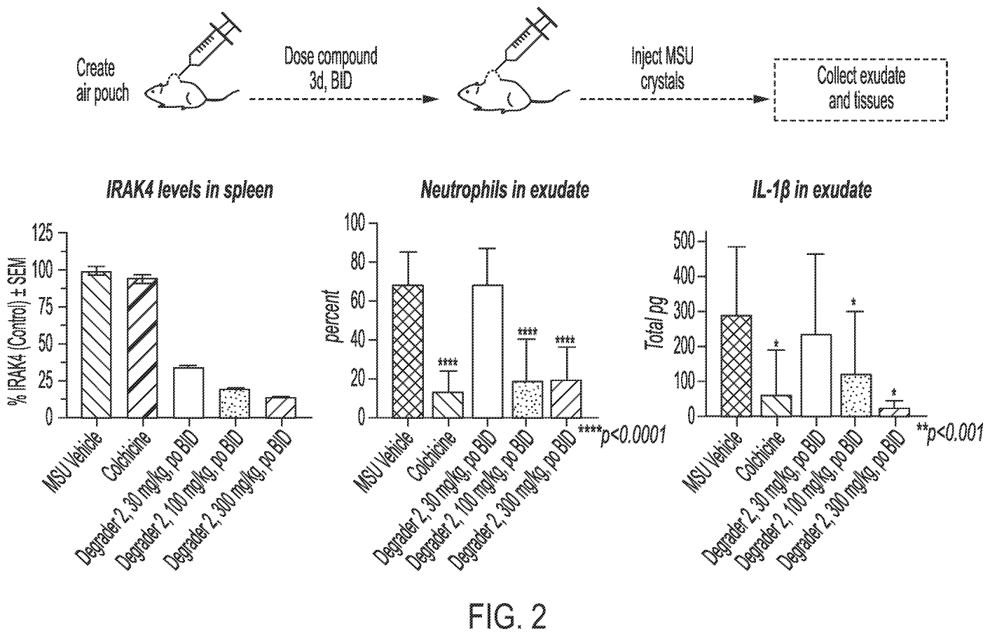

Degrader 2 was dosed orally for 3 days, BID following air pouch generation. On day 4, the last dose of compound was administered, and MSU crystals were injected into the air pouch. 12 hours later, relevant tissues and exudate from the pouch was collected. IRAK4 levels in spleen were measured by targeted mass spec. Neutrophil infiltrate counts were recorded, and IL-1βlevels were measured by ELISA from exudate. The results are shown in FIG. 2.

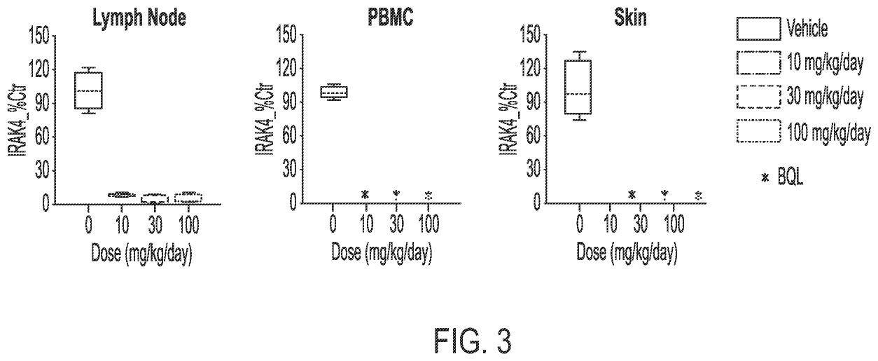

Degrader 2 was dosed orally, QD for 14 days in dog. 24 hours following last dose tissues were collected and IRAK4 levels were measured by targeted mass spec. The results are shown in FIG. 3.

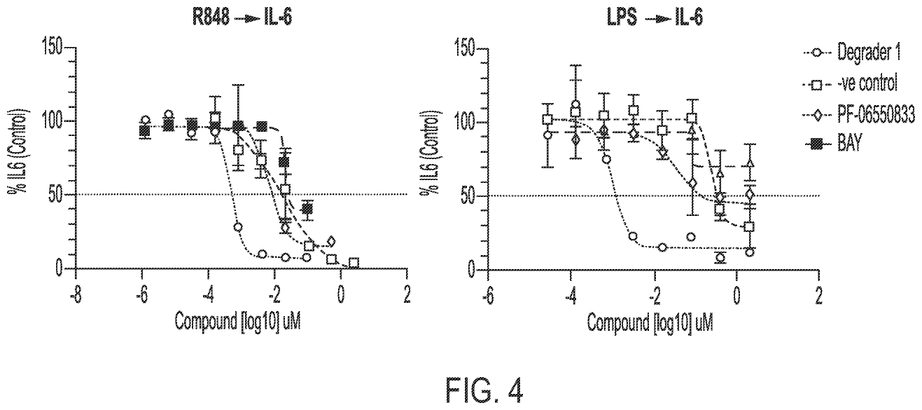

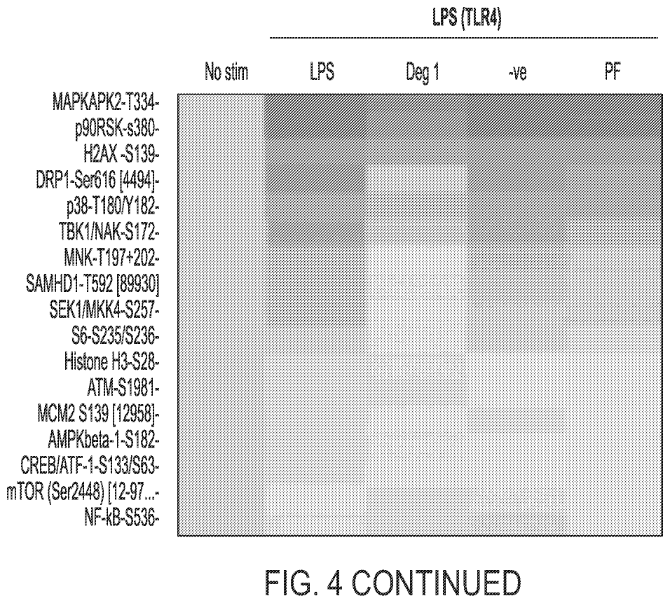

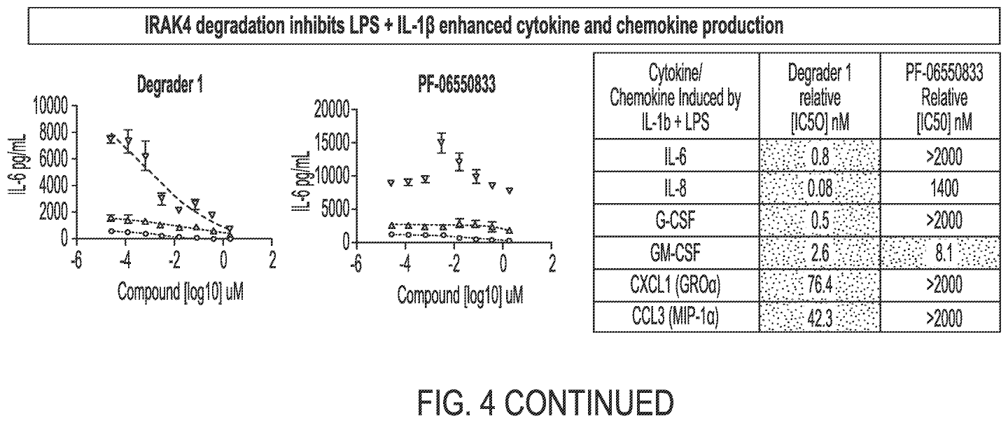

PBMCs were pre-treated with compounds for 20 hours followed by R848 (TLR7/8) or LPS (TLR4) stimulation. 5 hours post stimulation, cytokines were measured by MSD. For phosphoprotein profiling, samples were collected 15 min post stimulation. Flow methods were used to gate monocytes and measure phosphoproteins. The results are shown in FIG. 4.

PBMCs were pre-treated with compounds for 20 hours followed by dual activation with LPS at 10 ng/mL and IL-1b at 20 ng/mL. 24 hours following stimulation, cytokines were measured by MSD. The results are shown in FIG. 4.

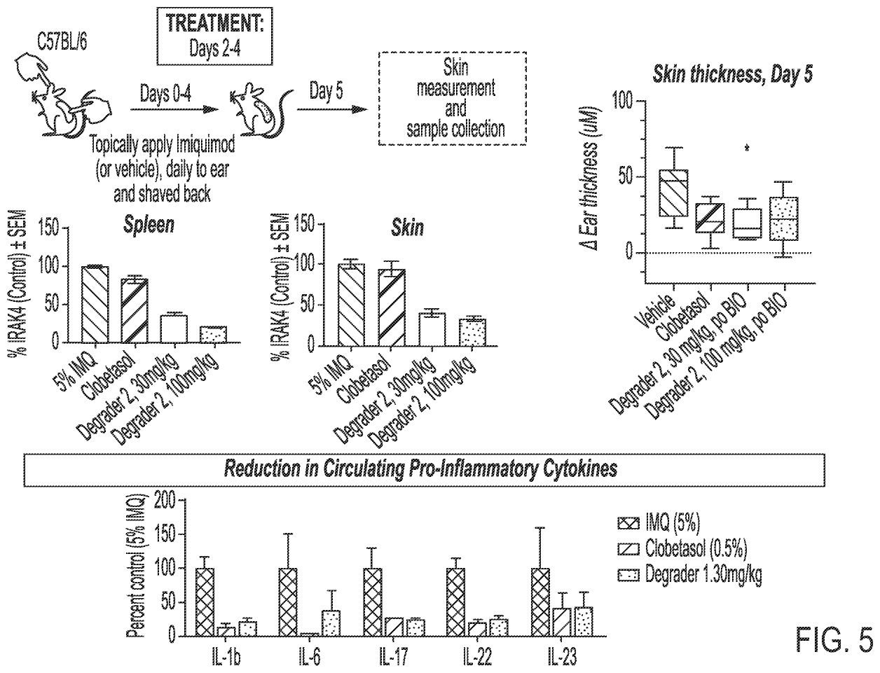

Imiquimod was applied to the ear on Day 0 and ear thickness was measured daily. Degrader 2 was dosed orally for 3 days, BID. At the end of the study (day 5) spleen and skin were collected and IRAK4 levels were measured by targeted mass spec. The results are shown in FIG. 5.

Degrader 1 was dosed i.p. for 3 days, BID. At the end of the study (day 5), plasma samples were collected and Pro-inflammatory cytokines were measured by Luminex assays. The results are shown in FIG. 5.

### CONCLUSION

IRAK4 degraders are highly effective and superior to SMI at inhibiting myddosome signaling and blocking cytokine/chemokine induction by TLR agonists and IL-1.

IRAK4 degraders are highly orally active in the mouse imiquimod psoriasis model, with reduction of skin thickening and both Th1 and Th17 cytokines. In addition, they effectively bock I1-1-driven neutrophilic inflammation in the mouse MSU air pouch model.

Daily oral dosing of an IRAK4 degrader in dogs for 2 weeks was well-tolerated and led to complete suppression of IRAK4 protein in skin and immune cells.

Collectively, these data show IRAK4 degraders have the potential to treat TLR/IL-1R-driven neutrophilic inflammation and autoimmune diseases such as hidradenitis suppurativa (HS).

While a number of embodiments of this invention are described, it is apparent that the examples may be altered to provide other embodiments that utilize the compounds and methods of this invention. Therefore, it will be appreciated that the scope of this invention is to be defined by the application and claims rather than by the specific embodiments that have been represented by way of example.

1. A method of treating hidradenitis suppurativa patient and/or atopic dermatitis in a patient having an elevated level of an inflammatory biomarker, comprising administering to the patient a therapeutically effective amount of an IRAK4 degrader.

2. A method of treating hidradenitis suppurativa patient and/or atopic dermatitis in a patient, comprising selecting a patient having an elevated level of an inflammatory biomarker, and administering to the patient a therapeutically effective amount of an IRAK4 degrader.

3. A method of treating hidradenitis suppurativa patient and/or atopic dermatitis in a patient, comprising measuring an inflammatory biomarker level in a sample of a patient, selecting a patient having an elevated level of an inflammatory biomarker, and administering to the patient a therapeutically effective amount of an IRAK4 degrader.

4. A method of treating hidradenitis suppurativa patient and/or atopic dermatitis in a patient having a reduced level of an inflammatory biomarker after a treatment with an IRAK4 degrader, comprising administering to the patient a therapeutically effective amount of an IRAK4 degrader.

5. A method of treating hidradenitis suppurativa patient and/or atopic dermatitis in a patient, comprising selecting a patient having a reduced level of an inflammatory biomarker after a treatment with an IRAK4 degrader, and administering to the patient a therapeutically effective amount of an IRAK4 degrader.

6. A method of treating hidradenitis suppurativa patient and/or atopic dermatitis in a patient, comprising measuring an inflammatory biomarker level in a sample of a patient after a treatment with an IRAK4 degrader, selecting a patient having a reduced level of an inflammatory biomarker after a treatment with an IRAK4 degrader, and administering to the patient a therapeutically effective amount of an IRAK4 degrader.

7. A method of treating hidradenitis suppurativa patient and/or atopic dermatitis in a patient, comprising administering to a patient a therapeutically effective amount of an IRAK4 degrader, measuring an inflammatory biomarker level in a sample of a patient after a treatment with an IRAK4 degrader, selecting a patient having a reduced level of an inflammatory biomarker after a treatment with an IRAK4 degrader, and administering to the patient a therapeutically effective amount of an IRAK4 degrader.

8. The method of any one of claims 3, 6, and 7, wherein the sample is a blood sample or a skin sample.

9. The method of any one of claims 1-8, wherein the inflammatory biomarker is IRAK4 in circulating peripheral blood mononuclear cells (PBMC).

10. The method of claim 9, wherein the circulating PBMC is selected from B cells, CD4−/CD8− (double-negative, DN) T cells, CD4+ T cells, CD8+ T cells, monocytes, and NK cells.

11. The method of any one of claims 1-8, wherein the inflammatory biomarker is a chemokine selected from CCL2, CCL20, CSF3, CXCL1, CXCL2, CXCL6, CXCL8, CXCL11, and CXCL13.

12. The method of any one of claims 1-8, wherein the inflammatory biomarker is selected from GZMB and PRF1.

13. The method of any one of claims 1-8, wherein the inflammatory biomarker is a cytokine selected from IFNG, IL10, IL1B, IL32, IL36G, IL6, IRF7, SOCS3, and TNF.

14. The method of any one of claims 1-8, wherein the inflammatory biomarker is a cytokine receptor selected from IL2RA, IL2RB, and IL18RAP.

15. The method of any one of claims 1-8, wherein the inflammatory biomarker is selected from MYD88, TLR1, TLR2, TLR3, TLR4, TLR6, TLR8, and TLR9.

16. The method of any one of claims 1-8, wherein the inflammatory biomarker is selected from NLRP3 and PTGS2.

17. The method of any one of claims 1-8, wherein the inflammatory biomarker is selected from CXCL6, CXCL8, CXCL1, CGAS, SOCS3, CXCL13, and CTSL.

18. The method of any one of claims 1-3, wherein the elevated level of the inflammatory biomarker refers to that the amount or concentration of the inflammatory biomarker in the patient is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to the amount or concentration of the inflammatory biomarker in a healthy person.

18. The method of any one of claims 4-7, wherein the reduced level of the inflammatory biomarker refers to that the amount or concentration of the inflammatory biomarker in the patient is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, after a treatment with an IRAK4 degrader.

19. The method of any one of claims 1-18, wherein the IRAK4 degrader is Degrader 1: 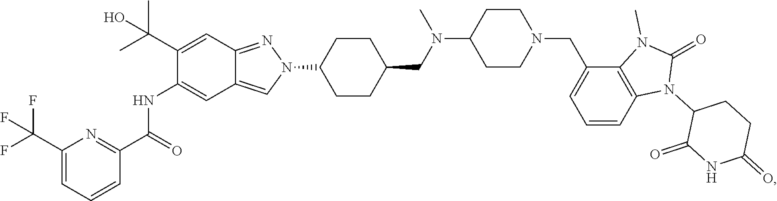
or a pharmaceutically acceptable salt thereof, or Degrader 2: 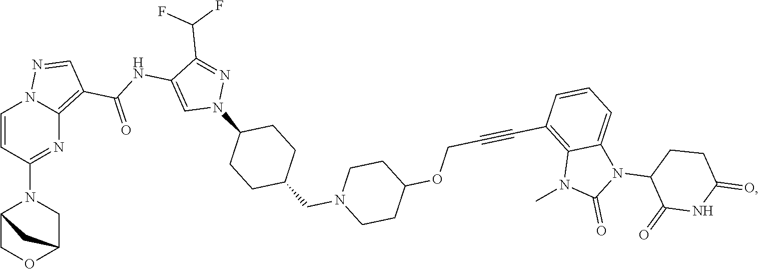 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]
or a pharmaceutically acceptable salt thereof.
